(19) United States
(12) Patent Application Publication
(10) Pub. No.: US 2026/0182726 A1
(43) Pub. Date: Jul. 2, 2026

(54) INTEGRATED MULTILAYERED SKINCARE MASK FOR SEQUENTIAL TREATMENT DELIVERY AND SKINCARE MASK UNDERLAYER

(71) Applicant: Glogenix LLC, Alameda, CA (US)

(72) Inventors: Yulia Casey, Alameda, CA (US); Richelle Lyndon, Pleasanton, CA (US)

(21) Appl. No.: 19/431,797

(22) Filed: Dec. 23, 2025

Related U.S. Application Data
(60) Provisional application No. 63/739,357, filed on Dec. 27, 2024.
(60) Provisional application No. 63/739,361, filed on Dec. 27, 2024.

(51) Int. Cl. A45D 44/00; A61K 8/02; A61Q 19/00

(52) U.S. Cl. CPC A45D 44/002; A61K 8/0212; A61Q 19/00

(57) ABSTRACT

The present disclosure describes a skincare mask comprising at least one humectant or hydrophilic layer and at least one at least partially occlusive layer, wherein the at least one humectant or hydrophilic layer and the at least one at least partially occlusive layer are arranged in a predefined spatial order that structurally encodes a predetermined sequence of ingredient exposure to skin upon a single application of the skincare mask. A skincare mask underlayer comprising at least one humectant or hydrophilic layer is also described. Methods for producing such a skincare mask and skincare mask underlayer are also disclosed.

### CROSS REFERENCE TO RELATED APPLICATIONS

This application is a Utility application which claims the benefit of priority to (1) U.S. Provisional application No. 63/739,357, filed Dec. 27, 2024, entitled “INTEGRATED MULTILAYERED SKINCARE MASK FOR SEQUENTIAL TREATMENT DELIVERY”; and (2) U.S. Provisional application No. 63/739,361, filed Dec. 27, 2024, entitled “TONING AND/OR PRIMING DISSOLVING UNDERLAYER”, each of which are hereby incorporated by reference in their entirety.

### FIELD OF THE INVENTION

The present disclosure relates generally to skincare products and methods of making the same. More specifically, this disclosure relates to multi-layered skincare sheet masks designed to deliver a sequential multi-step skincare regimen in a single application, and methods of making and using the same. The disclosure also relates to a skincare mask underlayer and methods of making and using the same.

### BACKGROUND OF THE INVENTION

Skincare routines have long been recognized as an important aspect of personal care and beauty regimens. Traditionally, these routines involve multiple steps, each designed to address specific skin concerns and provide various benefits. A typical multi-step skincare routine may include cleansing, toning, applying essence, using serums or treatments, and moisturizing. Each of these steps serves a distinct purpose in preparing, treating, and protecting the skin.

While comprehensive skincare routines can be highly effective, they often require significant time and effort to complete. This can be particularly challenging for individuals with busy lifestyles or those who frequently travel. The need to carry multiple products and dedicate time to apply them in a specific order can be inconvenient and may lead to inconsistent skincare practices. There are numerous problems associated with known skincare routines and products. For instance, order of application errors (Human Error): many skincare actives require specific sequencing. In the real world, users apply products in the wrong order, skip steps, or reverse layers unintentionally. Incorrect sequencing of skincare products can materially reduce performance due to premature occlusion, ph neutralization, or hinder penetration of active ingredients. This results in reduced efficacy, inactivation of actives and increased irritation.

There is commonly ingredient incompatibility across brands (Formulation systems problem): users combine products from different brands with incompatible solvents, destabilizing preservatives, and conflicting penetration enhancers. This leads to degradation, reduced bioavailability, and irritation.

There can be physical & cognitive accessibility (a real world constraint-elderly, disability, fatigue): multi-step routines require memory of order and timing, fine motor control, grip strength which put elderly users, post-procedure patients, and people with neurological conditions at disadvantage.

There can be inconsistent dosing and over/under application: with multiple products, users apply variable quantities and dosing it inconsistently day to day that can lead to irritation (retinoids, acids), underdosing of actives and non-reproducible outcomes.

There can be cross-interference during simultaneous application: when multiple liquid products are layered, solvents intermix immediately, uncontrolled diffusion occurs and occlusives may alter evaporation and diffusion behavior, potentially altering the penetration kinetics of active ingredients.

There can be time-dependent instability of actives: certain actives degrade rapidly when applied (retinoids oxidize, peptides hydrolyze, antioxidants lose activity). The known extended routines thus increase air and light exposure.

There can be skin barrier overload and irritation: sequential application of many products repeatedly can disrupt the barrier.

There can be packaging, storage and sustainability constraints: multiple products require multiple containers, preservatives, transport volume and increased waste and cost to the manufacturer and user.

Sheet masks have gained popularity in recent years as a convenient and portable skincare option. These masks are typically single-use products that deliver concentrated ingredients to the skin. However, most conventional sheet masks focus on a single aspect of skincare, such as hydration or brightening, rather than providing a comprehensive treatment. Conventional sheet masks are typically single-phase systems in which all ingredients are delivered simultaneously from a uniform substrate saturated with a single formulation. As a result, such masks are generally limited to addressing a single skincare objective, such as hydration or soothing, because combining multiple functional actives within one formulation presents technical challenges, including ingredient incompatibility, instability, uncontrolled release, and functional dilution.

The skincare industry has recognized the need for more efficient and user-friendly products that can deliver the benefits of a multi-step routine in a more convenient format. Various attempts have been made to combine multiple skincare steps into a single product or to create travel-friendly skincare sets. However, these solutions often still require separate application steps or fail to fully replicate the systematic approach of a traditional skincare routine. For instance, when multiple steps are combined, compromises in formulation stability and ingredient compatibility may occur, reducing overall efficacy. Current all-in-one solutions are formulated in liquid form and are not fast-absorbing. Therefore, they do not fully address the need for a streamlined, integrated, and portable system that is truly time-saving compared to a traditional skincare routine. Moreover, bundled or travel-sized products still require users to perform multiple application steps, waiting for absorption between steps and correctly sequence products, thereby preserving the same sources of inconsistency, human error and time burden as full-sized routines.

There remains a need for innovative skincare products that can effectively combine the benefits of a multi-step routine with the convenience of a single application. Such products could potentially improve skincare compliance, save time, and provide a more accessible approach to comprehensive skin treatment. The present invention intends to address and/or overcome the limitations discussed above by presenting new designs and methods not hitherto contemplated nor possible by known constructions.

### SUMMARY OF THE INVENTION

According to an aspect of the present invention, there is provided a skincare mask comprising at least one humectant or hydrophilic layer and at least one at least partially occlusive layer, wherein the at least one humectant or hydrophilic layer and the at least one at least partially occlusive layer are arranged in a predefined spatial order that structurally encodes a predetermined sequence of ingredient exposure to skin upon a single application of the skincare mask.

The predetermined sequence of ingredient exposure may be independent of user application behavior. A pre-engineered multilayer structure enforces correct order by design, independent of user behavior, thereby minimizing if not eradicating human error regarding application order of ingredients. Since all layers may be co-formulated and designed to be mutually compatible, this addresses the problem of ingredient compatibility across brands, for instance. In accordance with the invention, it may be that a single application format reduces steps, eliminates sequencing decisions and removes manual dexterity requirements that less-abled persons can otherwise experience. Techniques compatible with the present invention including electrospinning produce electrospun layers which allow pre-defined mass per layer, controlled active loading and repeatable exposure per use, thereby avoiding inconsistent dosing and over/under application that can occur with known products and methods. The skincare mask can separate layers that prevents premature intermixing and enables time-ordered interaction with the skin, which avoids cross-interference of different products that can occur with known products and methods. The skincare mask can retain the actives that are protected within fibers until dissolution and reduce time between exposure to absorption, thereby reducing or avoiding the problem of time-dependent instability of actives in known products and methods. The skincare mask can provide controlled layering allowing reduced mechanical rubbing, thereby avoiding or minimizing the problem of skin barrier overload and irritation experienced by use of known products and methods. Further, according to the invention, the skincare mask is operable to provide a single, dry, and compact format reducing packaging, improving portability, and lowering environmental footprint.

The skincare mask system provides a structured, multifunctional skincare platform in which ingredients are physically separated into distinct (optionally electrospun) layers having different compositions and dissolution behaviors. This architecture enables staged and sequential delivery of skincare actives, allows isolation of otherwise incompatible ingredients, and permits independent optimization of each layer for hydration, active delivery, or barrier support. Accordingly, the disclosed skincare mask (multilayer mask) delivers comprehensive skincare benefits within a single application, which cannot be achieved by conventional single-layer sheet masks or liquid-saturated substrates.

The skincare mask system is operable to integrate multiple functional skincare steps into a single step by physically separating ingredients into distinct (optionally electrospun) layers with defined compositions. This approach preserves the systematic logic of traditional routine—such as hydration preceding active delivery—while eliminating the need for multiple products or user-dependent sequencing, thereby enabling efficient, reproducible and comprehensive skincare in a single application. The skincare mask system is able to provide predefined compositions, enabling controlled, sequential delivery following a single placement on the skin.

In conventional routines, toning and hydration are typically performed prior to delivery of concentrated actives, followed by application of emollient or occlusive products to support barrier function. By spatially separating these functions into distinct layers, the disclosed mask preserves this functional logic while eliminating the need for multiple products or repeated user intervention. This approach improves reproducibility, reduces user error, and enables consistent delivery of skincare benefits across uses.

Incorporating humectants together with semi-occlusive or occlusive ingredients in a multilayered skincare mask offers a key advantage in that it mimics the natural sequence of skin hydration and barrier protection observed in effective skincare regimens. Depending on the desired active ingredient loading, one or more skincare actives may be incorporated into additional subsequent layers of the multilayered skincare mask once an active ingredient exceeds the loading capacity of a single layer, beyond which mechanical integrity, processability and dissolution behavior may be compromised.

Unlike conventional sheet masks, which consist of a uniform substrate saturated with a single liquid formulation, and unlike known electrospun sheet masks in which layers primarily function as passive carriers and dissolve substantially simultaneously, the disclosed skincare mask comprises multiple physically discrete electrospun nanofiber layers, each having a distinct composition and functional role. The layers are arranged in a predefined spatial order prior to application, such that ingredient sequencing is structurally encoded into the product rather than dependent on user application behavior. Through layer-specific compositions and spatial arrangement, the disclosed system enforces a predetermined sequence of skin exposure, enabling controlled, staged delivery.

The inventors understand that, although all layers rapidly hydrate, a transiently staggered release profile can be obtained under fast-dissolution conditions. The skin-facing layers, enriched in more hydrophilic and/or low molecular weight active ingredients preferentially contribute to the initial mass flux due to greater aqueous solubility and rapid dissolution at the skin interface. In contrast, more hydrophobic and/or higher molecular weight active ingredients localized in posterior layers exhibit slower matrix wetting and increased diffusional resistance.

While certain embodiments include two primary functional layers-such as a humectant-rich layer and an emollient or occlusive-rich layer-additional layers may be incorporated to accommodate higher active loading, isolate incompatible ingredients, or modulate dissolution timing without altering the fundamental delivery mechanism. Accordingly, the disclosed system is not limited to a specific number of layers, but rather to a plurality of layers arranged to provide controlled, sequential delivery.

The at least one humectant or hydrophilic layer may be positioned closer to a skin-contacting surface of the skincare mask than the at least one at least partially occlusive layer.

The at least one at least partially occlusive layer may be positioned outwardly relative to the at least one humectant or hydrophilic layer.

The at least one humectant or hydrophilic layer and the at least one partially occlusive layer may have different compositions.

The at least one humectant or hydrophilic layer and the at least one at least partially occlusive layer may have different dissolution behaviors upon exposure to moisture.

The layers may be configured to isolate incompatible ingredients in separate layers prior to use.

The layers may comprise physically discrete layers.

The at least one humectant or hydrophilic layer and at least one at least partially occlusive layer may comprise nanofiber layers.

The nanofiber layers may be electrospun nanofiber layers.

The at least one humectant or hydrophilic layer may be configured to dissolve more rapidly upon application to skin than the least one at least partially occlusive layer.

The at least one humectant or hydrophilic layer may be configured to attract and bind water molecules for increasing skin hydration upon application.

The at least one at least partially occlusive layer may be configured to reduce transepidermal water loss (TEWL). It may be that the at least one at least partially occlusive layer is configured to generate a skin barrier reinforcement.

The skincare mask may comprise two primary functional layers, including: a humectant-rich layer for use in skin hydration; and an emollient and/or at least partially occlusive-rich layer for use in skin barrier protection.

The skincare mask may comprise at least four functional layers, including: a toning layer; an essence layer; a serum layer; and a moisturizer layer.

At least one of the layers may further comprise an additive configured to improve electrospinning continuity, nanofiber mechanical integrity, and dissolution behavior.

The additive may comprise at least one penetration enhancer configured to modify solution properties during fabrication and/or nanofiber matrix properties after fabrication for improving electrospinning continuity, nanofiber uniformity, mechanical integrity, and dissolution behavior of layers. Penetration enhancing ingredients can improve the absorption of relatively hydrophilic active compounds into the deeper layers of the skin and enhance their potency.

Penetration-enhancing ingredients, when applied to the skin in combination with an active agent, facilitate, increase, or accelerate transport of the active agent into or across one or more layers of the skin, including the stratum corneum, epidermis, and/or dermis. Such enhancers may operate through various mechanisms, including disruption of the stratum corneum lipid matrix, improvement of active-compound solubility, and modification of skin protein structures to increase skin permeability.

Penetration enhancing ingredients may additionally function as plasticizers, humectants, solvents, and/or viscosity-modifying agents within the composition.

Such penetration enhancers may include, for example, polyols, glycols, alcohols, fatty acids, fatty alcohols, esters, cyclic acetals, alkyl glycoside, terpenes, surfactants, or combinations thereof, which may function to improve solubility of active ingredients, facilitate electrospinning or other fabrication processes, and enhance penetration of active ingredients into the skin. Non-limiting examples of penetration enhancers and/or solvent components include propanediol, butylene glycol, pentylene glycol, ethanol, isopropyl alcohol, polyethylene glycol, dimethyl isosorbide, oleic acid, linoleic acid, lauric acid, caprylic/capric triglycerides, isopropyl myristate, polysorbates, heptyl glucoside, lecithin, terpenes, or combinations thereof.

The at least one humectant or hydrophilic layer may comprise at least one of a toning layer, an essence layer, or a serum layer.

The at least one partially occlusive layer may comprise at least one of a serum layer or a moisturizing layer.

The at least one humectant or hydrophilic layer may be substantially a hydrating layer.

The at least one partially occlusive layer may comprise at least some hydrophobic material.

The at least one humectant or hydrophilic may have a thickness from about 5 μm to about 150 μm.

The at least one at least partially occlusive layer may have a thickness from about 5 μm to about 300 μm.

The at least one humectant or hydrophilic layer may comprise about 0.5-20 wt % of carrier polymer, about 0-15 wt % of co-polymer and/or polymer blend component, about 0.001-20 wt % of humectant, about 0-5 wt % of at least partially occlusive material, about 0.001-10 wt % of co-solvent electrospinning enhancer and/or penetration enhancer; and about 0-5 wt % of emollient.

The at least one partially occlusive layer may comprise about 0.5-20 wt % of carrier polymer, about 0-15 wt % of co-polymer and/or polymer blend component, about 0-15 wt % of humectant, about 0.001-20 wt % of at least partially occlusive material, about 0.001-10 wt % of co-solvent electrospinning enhancer and/or penetration enhancer; and about 0.001-20 wt % of emollient.

The toning layer may comprise Centella Asiatica, aloe vera, hyaluronic acid, ceramide, allantoin, chamomile, cucumber extract, green tea extract, ginseng, caffeine, licorice extract, beta glucan, oat extract, calendula, menthol, peppermint, hydrolyzed collagen, cetyl hexapeptide-8, lactic acid, mandelic acid, gluconolactone, niacinamide, hydroxypropyl bis-hydroxyethyldimonium chloride, maltodextrin, amino acids, amino acid derivatives, or combinations thereof. The toning layer is configured to provide initial hydration, calming, and barrier-conditioning upon contact with the skin, thereby preparing the skin surface for subsequent delivery of actives from one or more posterior layers. Ingredients in the toning layer are selected for their high aqueous solubility, low molecular weight, rapid dissolution behavior, and compatibility with electrospun fiber matrices, enabling fast wetting and dissolution at the skin interface. In particular, botanical extracts and humectants such as Centella Asiatica, aloe vera, beta-glucan, oat extract, and hyaluronic acid promote hydration and barrier comfort, while anti-inflammatory or soothing agents such as allantoin, chamomile, calendula, and licorice extract reduce irritation and support skin tolerance. Lactic acid, mandelic acid, and gluconolactone, which aid in mild exfoliation and surface renewal. Amino acids, amino acid derivatives, and hydrolyzed collagen contribute to osmotic balance and skin conditioning, while peptides such as cetyl hexapeptide-8 may support skin appearance without interfering with subsequent active delivery. The toning layer may further include carrier or matrix-support components, such as maltodextrin or other saccharides, which facilitate electrospinning, enhance fiber integrity, and promote rapid dissolution without leaving residue on the skin. In certain embodiments, the toning layer includes Centella Asiatica, aloe vera, hyaluronic acid, beta-glucan, allantoin, and amino acids dispersed within an electrospun polymer matrix, providing rapid hydration and skin conditioning upon application.

The essence layer may comprise fermented ingredients, antioxidants, vitamins, plant extracts, fruit extracts, herbal extracts, or combinations thereof.

The essence layer may comprise Galactomyces ferment, Saccharomyces extract, Bifida ferment lysate, Aspergillus ferment, niacinamide, resveratrol, coenzyme Q10, vitamin C, vitamin E, agave extract, algae extract, bilberry extract, cucumber extract, ginger root extract, grapeseed extract, honey extract, sea buckthorn extract, orange peel extract, saffron extract, purslane extract, pine bark extract, bamboo extract, avocado peptide, camellia seed extract, hydroxypropyl bis-hydroxyethyldimonium chloride, or combinations thereof. The essence layer is configured to provide intermediate skin conditioning and biochemical support following initial hydration provided by the toning layer and prior to delivery of more concentrated actives from subsequent layers. Ingredients in the essence layer are selected to be biologically active yet well-tolerated, and to function effectively in a hydrated skin environment established by earlier layer dissolution. Fermented ingredients may be preferred in the essence layer because fermentation can reduce molecular size, increase bioavailability, and enhance compatibility with electrospun fiber matrices, thereby facilitating efficient dissolution and skin interaction. Antioxidants and vitamins provide protection against oxidative stress and support skin homeostasis, while plant, fruit, and herbal extracts contribute complementary bioactive compounds that promote skin conditioning and resilience. In addition, ingredients in the essence layer can be selected to be chemically and functionally compatible with both preceding and subsequent layers, enabling isolation of sensitive actives while maintaining a continuous delivery sequence. By localizing these ingredients within a distinct essence layer, the disclosed system avoids premature interaction with occlusive components and enables controlled exposure under favorable skin conditions, which cannot be reliably achieved in single-phase or simultaneously dissolving formulations. In certain embodiments, the essence layer includes fermented botanical extracts in combination with antioxidants and vitamins, providing enhanced skin conditioning and biochemical support prior to active delivery. Fermented ingredients such as Galactomyces, Saccharomyces, Bifida, and Aspergillus ferments may be selected for their enhanced bioavailability, reduced molecular complexity, and compatibility with electrospun fiber matrices. Antioxidants and vitamins, including niacinamide, resveratrol, coenzyme Q10, and vitamins C and E, support skin homeostasis and protection against oxidative stress under hydrated conditions. Plant, fruit, marine, and botanical extracts may be selected for their complementary bioactive profiles and chemical compatibility, enabling conditioning and resilience without destabilizing subsequent layers. Localization of these ingredients within a distinct essence layer allows controlled exposure under favorable skin conditions while minimizing premature interaction with occlusive components, thereby supporting effective sequential delivery within the multilayer architecture.

The serum layer may comprise alpha-arbutin, retinol, bakuchiol, DNA-derived compounds, salicylic acid, or combinations thereof. These ingredients may be used because they provide high-potency, function-driven skincare activity that benefits from delivery under controlled and conditioned skin environments. Alpha-arbutin, kojic acid, and tranexamic acid, retinol, bakuchiol, and salicylic acid are widely recognized for their effects on skin appearance, turnover, and tone, while DNA-derived compounds support skin conditioning and repair processes. However, such ingredients are often associated with sensitivity, instability, or irritation when applied without adequate skin preparation or when delivered simultaneously with occlusive components. By localizing these ingredients within a distinct serum layer positioned posterior to hydrating and conditioning layers, the disclosed skincare mask/system enables delivery under hydrated, buffered skin conditions, which can improve tolerance and consistency of exposure. Separation of these ingredients from earlier and later layers further reduces premature interaction, degradation, or irritation, enabling effective incorporation of potent actives within a single-application, multilayer architecture.

The skincare mask may further comprise a second serum layer. The inclusion of a second serum layer is beneficial when the total active ingredient loading exceeds the capacity of a single serum layer or when certain active ingredients exhibit incompatibility, stability limitations, or differing delivery requirements. By distributing serum actives across multiple layers, the disclosed system enables higher cumulative active loading while preserving mechanical integrity, electrospinning processability, and predictable dissolution behavior. In addition, separating serum actives into multiple layers allows isolation of ingredients with differing sensitivities, such as those requiring different microenvironments, dissolution timing, or exposure profiles. The second serum layer may also be configured to dissolve after an initial serum layer, thereby extending or modulating active exposure without requiring additional user application steps.

The moisturizing layer may comprise humectants, emollients, semi-occlusives, occlusives, or a combination thereof. In certain embodiments, the skincare mask/delivery system comprises at least two primary types of layers, including a humectant-rich layer and an emollient- and/or occlusive-rich layer, which together are sufficient to achieve controlled, sequential delivery and post-delivery stabilization. In other embodiments, these primary functional layers are further subdivided into multiple layers, such as toning, essence, serum, and moisturizing layers, to accommodate higher active loading, ingredient compatibility, or modulation of dissolution behavior. Accordingly, the invention is not limited to a specific number or naming of layers, but rather to the functional organization and spatial ordering of layers within the multilayer architecture.

The moisturizing layer may comprise glycerin, hyaluronic acid, polyglutamic acid, alpha-hydroxy acids, panthenol, betaine, ceramides, squalane, silicone-based ingredients (dimethicone, cyclomethicone, caprylyl methicone), Coco-caprylate/caprate, shea butter, cocoa butter, fatty acids, peptides, or combinations thereof. These ingredients may collectively support moisture retention, barrier reinforcement, and post-delivery stabilization following dissolution of upstream layers. Humectants such as glycerin, hyaluronic acid, polyglutamic acid, panthenol, and betaine attract and retain water within the stratum corneum, thereby sustaining hydration established by earlier layers. Emollients and lipophilic components, including ceramides, squalane, jojoba oil, argan oil, coco-caprylate/caprate, fatty acids, may help to fill spaces between skin cells, soften the skin, and reduce water loss. Peptides and alpha-hydroxy acids may further support skin conditioning and surface renewal when delivered within a moisturized, buffered environment, while localization of these ingredients within a terminal moisturizing layer minimizes premature occlusion that could otherwise interfere with delivery from upstream layers. Semi occlusives or occlusives may be incorporated into the moisturizing layer to reduce transepidermal water loss and enhance barrier integrity. Shea butter and cocoa butter may be selected for their nourishing properties. Silicone-based ingredients such as dimethicone, cyclomethicone, caprylyl methicone may be selected to reduce transepidermal water loss and maintain a non-greasy sensory profile. Accordingly, incorporation of these ingredients within the moisturizing layer enhances comfort, consistency, and durability of the single-application skincare system.

The one or more of the toning layer, the essence layer, the serum layer, and the moisturizing layer may further comprise a polymer. The polymer may be included to provide structural integrity, processability, and to control dissolution, release behavior, and overall structural configuration of the electrospun nanofiber layers. In particular, the polymer functions as a fiber-forming matrix that enables electrospinning of uniform nanofibers, binds and distributes active ingredients within each layer, and maintains mechanical cohesion during handling and application. In addition, polymer selection and composition influence wetting behavior, dissolution rate, and diffusional resistance, thereby contributing to the sequential delivery achieved by the multilayer architecture. Polymers further facilitate compatibility among ingredients, reduce premature aggregation or crystallization, and enable tuning of layer thickness and porosity without altering active composition. Polymers may also contribute to the overall feel and texture of the mask on the skin, potentially enhancing user comfort and experience.

The polymer may comprise denatured collagen, collagen peptides, gelatin, fermented collagen, polypropylene oxide, hydrolyzed protein, whey protein, soy protein, pullulan, polyethylene oxide, polyvinyl alcohol, poly(oxazoline), hydroxypropyl methyl cellulose, polycaprolactone, polylactic acid, polylactic glycolic acid, galactomannan, glucomannan, guar gum, locust bean gum, xanthan gum, carrageenan, alginate, chitosan, poloxamers, or combinations thereof. These polymers may provide fiber-forming capability, mechanical integrity, and tunable dissolution behavior suitable for electrospun nanofiber layers, depending on the intended function of each layer. Water soluble polymers may be selected to form layers with faster dissolution and to provide hydration. These include collagen, collagen peptides, gelatin, fermented collagen, hydrolyzed protein, whey protein, soy protein, pullulan, polyethylene oxide, polyvinyl alcohol, hydroxypropyl methyl cellulose, galactomannan, glucomannan, guar gum, locust bean gum, xanthan gum, carrageenan, alginate, and poly(oxazoline). Polymers with relatively low water solubility or hydrophobic character may be selected to form layers with slower dissolution, increased structural integrity, or barrier functionality. These include polycaprolactone (PCL), polylactic acid (PLA), polylactic-glycolic acid (PLGA), chitosan and polypropylene oxide (PPO). Protein- and polysaccharide-based polymers offer biocompatibility and rapid hydration. Selection among these polymers allows tailoring of layer strength, porosity, wetting behavior, and ingredient compatibility, thereby supporting reproducible fabrication and controlled delivery within the multilayer skincare architecture.

One or more of the at least one humectant or hydrophilic layer and at least one at least partially occlusive layer may be supported on a substrate.

The skincare mask may be fabricated as a free-standing multi-layered sheet. In some embodiments, the skincare mask or one or more individual layers may be supported on a substrate.

The substrate can be used to improve the integrity of the product and prevent damage during transport. It can improve the lifespan of the product by maintaining a dry packaging environment. In addition to providing mechanical support for thin (for instance, electrospun) multilayer sheets, these substrates may enhance comfort and skin conformity, promote long-term hydration and barrier protection, improve dissolution through enhanced spreadability, and offer environmentally friendly and sustainable characteristics, while being compatible with the skincare mask layers. In some embodiments, the substrate facilitates handling and uniform placement of the mask on skin without tearing or premature dissolution.

The substrate may comprise a hydrogel, biodegradable fabric, biodegradable polyester, a nonwoven material (spunbound, spunlace, meltblown), a polymeric film or sheet, a silicone-based sheet, a metallic foil, or combinations thereof.

The substrate may have a thickness from about 0.01 mm-5 mm.

In another aspect, the present invention comprehends a skincare mask as defined herein for use in: hydrating skin; supporting the skin barrier and reducing moisture loss; improving skin softness, texture, and overall appearance; enhancing delivery of skincare actives; and/or improving the cosmetic efficacy of skincare actives.

In another aspect, the present invention contemplates use of a skincare mask comprising at least one humectant or hydrophilic layer and at least one at least partially occlusive layer, wherein the use is for: hydrating skin; supporting the skin barrier and reducing moisture loss; improving skin softness, texture, and overall appearance; enhancing delivery of skincare actives; and/or improving the cosmetic efficacy of skincare actives.

In another aspect, the present invention envisages a method of using a skincare mask comprising at least one humectant or hydrophilic layer and at least one at least partially occlusive layer to cleanse skin, the method comprising the steps of: contacting the face with moisture or water; placing the skincare mask on the skin with a humectant or hydrophilic layer facing the skin and maintaining contact for a predetermined period to release skincare components while an at least partially occlusive layer reduces moisture loss; and removing the skincare mask after partial or complete dissolution of one or more layers of the skincare mask.

The method may comprise the step of: after removal of the skincare mask, wiping or rinsing the skin to remove residue or impurities, or leaving residual components on the skin for extended contact, optionally including overnight use.

In another aspect, the present invention encompasses a method of producing a skincare mask, comprising: fabricating at least one humectant or hydrophilic layer; fabricating at least one at least partially occlusive layer; and assembling the layers in a predefined spatial order such that the skincare mask provides the predetermined sequence of ingredients exposure to skin upon a single application.

The techniques described herein relate to a method of producing a skincare mask, including fabricating a plurality of electrospun nanofiber layers arranged in a predefined order, wherein the layers comprise at least a humectant-rich layer and an emollient- and/or occlusive-rich layer sufficient to achieve controlled, sequential delivery following a single application. In certain embodiments, the method further includes fabricating additional layers, such as a toning layer, an essence layer, one or more serum layers, and a moisturizing layer, to subdivide these primary functions, increase active loading, isolate incompatible ingredients, or modulate dissolution behavior. Each layer may be fabricated separately with a layer-specific composition and then stacked or deposited in a defined spatial order prior to application. Sequential administration on skin can be achieved by structural ordering and layer-specific wetting and dissolution characteristics, such that a skin-facing layer dissolves preferentially upon contact with skin moisture, while posterior layers experience delayed wetting and increased diffusional resistance. This produces a transient but reproducible sequence of ingredient exposure without requiring multiple user application steps. Conventional sheet masks, including single-layer and known multilayer masks, deliver ingredients simultaneously from a uniform substrate or from layers that dissolve substantially concurrently and therefore lack mechanisms to enforce order or timing of delivery. In contrast, the method of the invention may reside not in the absolute number of layers, but in the predefined order of fabrication and assembly, layer-specific compositions, and spatially enforced sequencing that collectively enable controlled, sequential administration within a single application.

Fabricating may comprise electrospinning, electrospraying, solvent casting, coating, freeze drying, hot melt extrusion, 3D printing, or combinations thereof. Electrospinning is an advantageous technique because it enables formation of uniform nanofiber layers with high surface area, tunable porosity, and controllable thickness, which facilitate rapid wetting, predictable dissolution, and incorporation of active ingredients within a fiber matrix. Electrospraying may be used to deposit particulates or coatings with controlled distribution, while solvent casting and coating provide scalable alternatives for forming continuous layers. Solvent casting or coating may also be used to form denser layers with slower dissolution or higher barrier properties. In at least some embodiments, freeze drying may be employed to preserve sensitive ingredients and generate porous structures, whereas hot melt extrusion and 3D printing enable solvent-free fabrication and precise spatial control of layer geometry. 3D printing may also be carried out using solvent based processes. Selection among these techniques allows tailoring of layer morphology, mechanical integrity, and dissolution behavior while maintaining the predefined spatial order required for sequential administration in a single application, based on the desired properties of each layer and compatibility with the layer compositions used therein. Combining multiple fabrication techniques may achieve varied release behaviors and differentiated architectures not readily attainable using a single fabrication method, such as zoned, patterned, or customized designs.

The method may comprise the step of: further comprising bonding the layers for preserving the predefined spatial order.

The method may comprise fabricating at least four functional layers, including a toning layer, an essence layer, a serum layer, and a moisturizing layer.

In another aspect, the present invention understands a method of producing a skincare mask, comprising: fabricating a first layer comprising a toner; fabricating a second layer comprising an essence; fabricating a third layer comprising a serum; and fabricating a fourth layer comprising a moisturizer.

The method may further comprise attaching the first layer to the second layer.

The method may further comprise attaching the second layer to the third layer.

The method may further comprise attaching the third layer to the fourth layer. The multilayered sheet can be assembled by sequential electrospinning different formulations onto the same collector or substrate. For example, the toner layer may be fabricated first, and the essence layer fabricated onto the toner layer, and so forth. Alternatively, individual layers can be fabricated separately using one or more techniques, such as electrospinning, solvent casting, coating, or other methods described herein, and subsequently combined through a lamination process to form a multilayer structure. This approach allows independent optimization of layer composition while maintaining mechanical integrity, layer alignment, and reproducible sequential delivery. The lamination may be achieved using pressure, heat, solvent vapor, biocompatible adhesives or combinations thereof to maintain mechanical integrity, minimize interference with the active ingredients in each layer, prevent delamination during handling and use. Ultrasonic welding may be used to fuse layers together via localized interfacial heating. In some embodiments, the bonding process may be tailored to achieve specific dissolution or release profiles for the multi-layered mask.

The skincare mask may be produced through a multi-step fabrication process involving the creation and assembly of individual layers. This approach may provide advantages including the absence of cross-interference between layers during fabrication, such as partial dissolution of a previously electrospun layer by residual or non-volatile solvents, and enables faster product iteration and production customization.

Fabricating may comprise suspending each of the toner, the essence, the serum, and the moisturizer ingredients in a polymer. Suspending by dispersing or dissolving the ingredients within the polymer (for instance, a polymer solution) facilitates homogeneous distribution of components that exhibit limited solubility and miscibility with the polymer matrix, and helps prevent phase separation both prior to and during the fabrication process. The suspension may be achieved through mechanical mixing, or other dispersion techniques to ensure uniform distribution of the active ingredients within the polymer matrix. In some embodiments, the polymer selection enables rapid dissolution of a toner layer, enhanced hydration of an essence layer, controlled or sustained release of a serum layer, and long-lasting hydration and barrier formation of a moisturizing layer, optionally with the inclusion of plasticizers, stabilizers, or other additives to tailor layer properties.

The polymer may comprise denatured collagen, collagen peptides, gelatin, fermented collagen, polypropylene oxide, hydrolyzed protein, whey protein, soy protein, pullulan, polyethylene oxide, polyvinyl alcohol, hydroxypropyl methyl cellulose, polycaprolactone, polylactic acid, polylactic glycolic acid, galactomannan, glucomannan, guar gum, locust bean gum, xanthan gum, carrageenan, alginate, chitosan, poly(oxazoline), poloxamers, or combinations thereof. The selection of the polymer may depend on the desired properties of each layer, such as dissolution rate, release profile, and compatibility with the active ingredients or with the specific components of each layer.

In another aspect, the present invention provides a skincare mask underlayer comprising at least one humectant or hydrophilic layer.

The underlayer may be configured to dissolve.

The underlayer may be configured to be used under a skincare mask.

The underlayer may be a priming layer.

The underlayer may be configured to prepare the skin for treatment, promote uniform mask contact, and/or enhance absorption and cosmetic efficacy of subsequently delivered skincare actives.

The skincare mask underlayer may comprise a toner.

The toner may comprise Centella Asiatica, aloe vera, hyaluronic acid, ceramide, allantoin, chamomile, cucumber extract, green tea extract, ginseng, caffeine, licorice extract, beta glucan, oat extract, calendula, menthol, peppermint, hydrolyzed collagen, fermented ingredients, antioxidants, vitamins, plant extracts, fruit extracts, herbal extracts, Galactomyces ferment, Saccharomyces extract, Bifida ferment lysate, Aspergillus ferment, niacinamide, resveratrol, Asqualane, panthenol, polyglutamic acid, amino acids, amino acid derivatives, glyceryl glucoside, acetyl hexapeptide-8, maltodextrin, orange peel extract, saffron extract, purslane extract, pine bark extract, bamboo extract, avocado peptide, bakuchiol, camellia seed extract, coenzyme Q10, vitamin C, vitamin E, agave extract, algae extract, bilberry extract, cucumber extract, ginger root extract, grapeseed extract, honey extract, sea buckthorn extract, lactic acid, mandelic acid, gluconolactone, hydroxypropyl bis-hydroxyethyldimonium chloride, or combinations thereof.

The skincare mask underlayer may further comprise a polymer.

The polymer may comprise denatured collagen, collagen peptides, gelatin, fermented collagen, polypropylene oxide, hydrolyzed protein, whey protein, soy protein, pullulan, polyethylene oxide, polyvinyl alcohol, hydroxypropyl methyl cellulose, polycaprolactone, polylactic acid, polylactic glycolic acid, galactomannan, glucomannan, guar gum, locust bean gum, xanthan gum, carrageenan, alginate, chitosan, poly(oxazoline), poloxamers, or combinations thereof.

The toning layer may be released upon exposure to a skincare mask.

According to another aspect, the present invention contemplates a method of manufacturing a skincare mask underlayer, comprising: fabricating at least one humectant or hydrophilic layer by a technique comprising electrospinning, electrospraying, solvent casting, coating, freeze drying, hot melt extrusion, 3D printing, or combinations thereof. The at least one humectant or hydrophilic layer may comprise a toner.

The method may further comprise suspending the toner in a polymer.

The polymer may comprise denatured collagen, collagen peptides, gelatin, fermented collagen, polypropylene oxide, hydrolyzed protein, whey protein, soy protein, pullulan, polyethylene oxide, polyvinyl alcohol, hydroxypropyl methyl cellulose, polycaprolactone, polylactic acid, polylactic glycolic acid, galactomannan, glucomannan, guar gum, locust bean gum, xanthan gum, carrageenan, alginate, chitosan, poly(oxazoline), poloxamers, or combinations thereof.

In some embodiments, the skincare mask may be configured for application to the face or neck. In further embodiments, the skincare mask may be adapted for application to other skin surfaces of a user, including hands, arms, legs, torso, or other body areas. The skincare mask may be provided in various shapes and configurations, including anatomically contoured shapes, geometric shapes, patch-like forms, strip-like forms, or segmented configurations having zones with different layer compositions.

The dissolution rates, release profiles, and overall efficacy of the skincare of each layer in the skincare mask may be controlled through various means. In some embodiments, dissolution behavior may be adjusted by changing the incorporated ingredients or layer composition, including polymer type, polymer-to-active ratio, ingredient concentration, ingredient selection, inorganic or organic fillers, crosslinkers, layer thickness, and layer morphology, may influence fabrication behavior and release characteristics by affecting solution properties, porosity, and structural cohesion, thereby modulating dissolution rate and release profile of active ingredients. In some embodiments, the morphology of one or more layers may be modified by enhancing fiber fusion or inter-fiber bonding to reduce porosity, thereby increasing structural cohesion and slowing dissolution and release of active ingredients.

The incorporation of polymers into the skincare mask layers may allow for the development of advanced delivery systems. In some embodiments, stimuli-responsive polymers or ingredients may be used to create layers that respond to specific environmental cues, such as temperature or pH changes, or the presence of specific compounds on the skin surface, to release active ingredients at targeted times or locations on the skin.

The embodiments of the present disclosure may be combined as appropriate to form new embodiments.

### BRIEF DESCRIPTION OF THE DRAWINGS

These and other aspects and features of the present invention will become apparent to those ordinarily skilled in the art upon review of the following description of specific embodiments of the invention in conjunction with the accompanying figures, wherein:

FIGS. 1a and 1b are schematic representations of a skincare mask in accordance with an embodiment of the invention in which FIG. 1a shows a top view and FIG. 1b shows a sectional side view through A-A;

FIGS. 2a to 2c are schematic side view representations of a skincare mask in accordance with alternative embodiments of the invention;

FIG. 3 is a schematic side view representation of a skincare mask in accordance with an embodiment of the invention including a substrate;

FIG. 4 is a schematic side view representation of a skincare mask in accordance with an embodiment of the invention illustrating dissolution rates;

FIGS. 5A to 5d are schematic front view representations of a user using a skincare mask in accordance with an embodiment of the invention in which FIG. 5a shows a user's face being moistened, FIG. 5b shows a user's face applied with a skincare mask, FIG. 5c shows a user's face with the skincare mask removed, and FIG. 5d shows a user's face optionally being rinsed;

FIG. 6 is a flow diagram illustrating methods for fabricating a multilayer skincare mask in accordance with embodiments of the present invention;

FIG. 7a is a schematic side view representation of a skincare mask underlayer in accordance with an embodiment of the invention;

FIG. 7b is a schematic side view representation of a skincare mask underlayer in accordance with another embodiment of the invention;

FIG. 8 is a schematic side view representation of a skincare mask underlayer in accordance with an embodiment of the invention being configured/arranged relative to a skincare mask; and

FIG. 9 is SEM images of electrospun nanofiber sheets containing 5-7 wt % PVA in the presence of 1,3-propanediol exhibited minimal to no bead formation and no observable fiber fusion.

Aspects, features, benefits, and advantages of the embodiments described herein will be apparent with regard to the following description, appended claims, and accompanying drawings where:

### DETAILED DESCRIPTION OF THE INVENTION

Before compounds, compositions and methods are described in detail, it is to be understood that this disclosure is not limited to the particular processes, compositions, or methodologies described, as these may vary. It is also to be understood that the terminology used in the description is for the purpose of describing the particular versions or embodiments only, and is not intended to limit the scope of the disclosure which will be limited only by the appended claims. Unless defined otherwise, all technical and scientific terms used herein have the same meaning as commonly understood by one of the ordinary skill in the art. Although any methods and materials similar or equivalent to those described herein can be used in the practice or testing of embodiments of the disclosure, the preferred methods, devices, and materials are now described.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

As used herein, the term “about” means plus or minus up to 20% of the numerical value of the number with which it is being used. For example, “about 50%” means in the range of 40-60% and includes exactly 50%. The term “about” may refer to plus or minus 1%, 5%, 10%, 15%, or 20% of the numerical value of the number with which it is being used.

As used herein, unless specifically indicated, the term “active ingredient” refers to a compound of any of the formulae as described herein, particularly those having a specific therapeutic or beneficial effect.

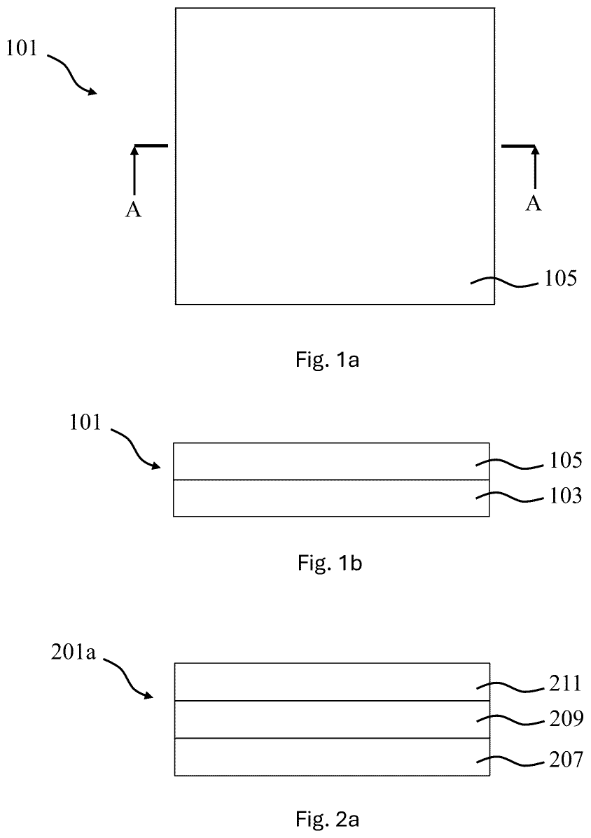

FIG. 1a is a plan view representation of a multilayered skincare mask 101, formed according to an embodiment of the present disclosure. As shown, in this embodiment, the skincare mask 101 has a square shape, but other shapes are also contemplated within the scope of this disclosure that can facilitate the function of the skincare mask 101. An at least one at least partially occlusive layer 105 is visible in this plan view since this is the uppermost layer in this configuration.

FIG. 1b shows a sectional side view through A-A (shown in FIG. 1a) of the multilayered skincare mask 101. The skincare mask 101 comprises at least one humectant or hydrophilic layer 103 and at least one at least partially occlusive layer 105, wherein the at least one humectant or hydrophilic layer 103 and the at least one at least partially occlusive layer 105 are arranged in a predefined spatial order that structurally encodes a predetermined sequence of ingredient exposure to skin upon a single application of the skincare mask 101. In this embodiment, the predetermined sequence of ingredient exposure is independent of user application behavior. As it can be seen, in this embodiment, the at least one humectant or hydrophilic layer 103 is positioned at the bottom so it is intended to be closer to a skin-contacting surface of the skincare mask 101 than the at least one at least partially occlusive layer 105 which is at the top of the stacked arrangement. In this embodiment, the at least one humectant or hydrophilic layer 103 comprises at least one of a toning layer, an essence layer, or a serum layer (not shown); more particularly, the at least one humectant or hydrophilic layer 103 comprises about 0.5-20 wt % of carrier polymer, about 0-15 wt % of co-polymer and/or polymer blend component, about 0.001-20 wt % of humectant, about 0-5 wt % of at least partially occlusive material, about 0.001-10 wt % of co-solvent electrospinning enhancer and/or penetration enhancer; and about 0-5 wt % of emollient. In this embodiment, the at least one partially occlusive layer 105 comprises at least one of a serum layer or a moisturizing layer (not shown); more particularly, the at least one partially occlusive layer 105 comprises about 0.5-20 wt % of carrier polymer, about 0-15 wt % of co-polymer and/or polymer blend component, about 0-15 wt % of humectant, about 0.001-20 wt % of at least partially occlusive material, about 0.001-10 wt % of co-solvent electrospinning enhancer and/or penetration enhancer; and about 0.001-20 wt % of emollient.

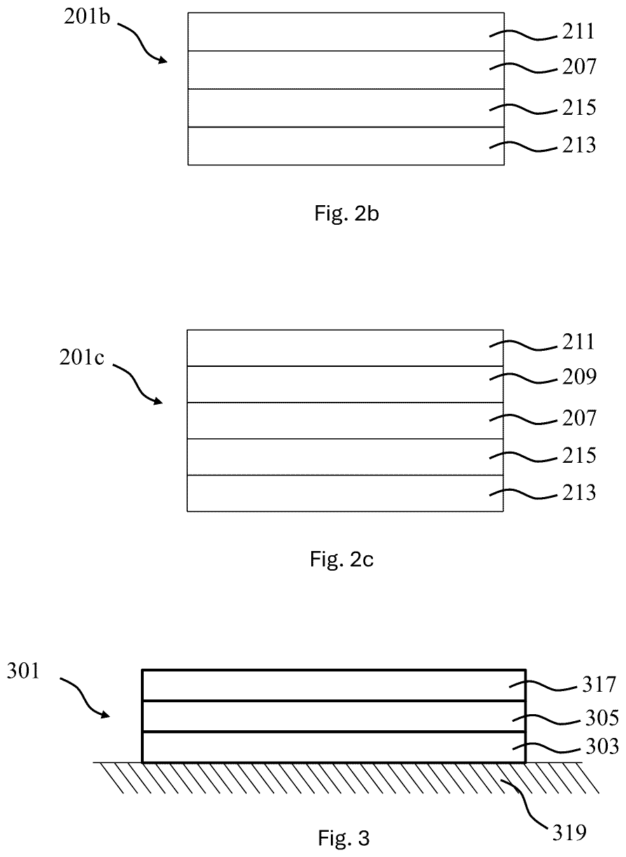

Referring now to FIGS. 2a to 2c, there are shown schematic side view representations of a skincare mask in accordance with alternative embodiments of the invention.

The embodiment of FIG. 2a is a three-layer skincare mask 201a which comprises a serum 1 layer 207, a serum 2 layer 209, and a moisturizing layer 211. They are in a stacked arrangement with the serum 1 layer 207 being the proximal skin-contacting surface, with the moisturizing layer 211 being the distal surface. The ingredients/contents of each of these layers are as defined herein.

The embodiment of FIG. 2b is a four-layer skincare mask 201b which comprises a toner layer 213, an essence layer 215, a serum layer 207, and a moisturizing layer 211. They are in a stacked arrangement with the toner layer 213 being the proximal skin-contacting surface, with the moisturizing layer 211 being the distal surface. The ingredients/contents of each of these layers are as defined herein.

The embodiment of FIG. 2c is a five-layer skincare mask 201c which comprises a toner layer 213, an essence layer 215, a serum 1 layer 207, a serum 2 layer 209, and a moisturizing layer 211. They are in a stacked arrangement with the toner layer 213 being the proximal skin-contacting surface, with the moisturizing layer 211 being the distal surface. The ingredients/contents of each of these layers are as defined herein.

In some embodiments, the skincare mask of the invention can further include a second serum layer or multiple additional serum layers between a serum layer and a moisturizing layer. In some embodiments, the skincare mask can include a second serum layer in place of a moisturizing layer. In some embodiments, the skincare mask does not include a toning layer and instead includes one or more serum layers, one or more essence layers, and a moisturizing layer. For example, in some embodiments, the skincare mask can include an essence layer, three serum layers, and a moisturizing layer. Other configurations of the presently disclosed layers are also contemplated. Depending on the desired active ingredient loading, one or more skincare actives may be incorporated into additional subsequent layers of the multilayered skincare mask once an active ingredient exceeds the loading capacity of a single layer, beyond which mechanical integrity, processability and dissolution behavior may be compromised. The use of a second serum layer in place of the moisturizing layer does not imply that a moisturizing function is absent. The serum layer may contain semi-occlusive components that provide moisturizing or barrier effects, such that the functional role of a moisturizing layer is still achieved. The skincare delivery system can include at least two functional layer types, comprising a humectant-rich layer and a semi-occlusive or occlusive-rich layer. One or more of the layers may optionally include multiple sublayers for incorporation of additional active agents while maintaining ingredient stability through layer separation. Traditionally, best practices for layering multiple skincare products prioritize enhancing skin hydration by maximizing water retention and barrier function, applying from the thinnest to the thickest product consistencies. Inadequate layering of multiple skincare products can lead to skin irritation and disruption of the skin barrier, particularly in individuals with sensitive or compromised skin. For example, applying retinol directly onto bare skin without proper buffering may result in localized redness or peeling due to excessive penetration of the active compound. Likewise, the premature application of occlusive-rich formulations prior to water-based moisturizers may diminish overall hydration efficacy, as the occlusive layer hinders the absorption of humectants and water-soluble actives. When humectants are used without an accompanying occlusive agent, they may promote increased transepidermal water loss, particularly from compromised skin in low-humidity environments. Such ineffective layering practices may therefore compromise both skin comfort and the overall efficacy of skincare routines.

Referring now to FIG. 3, there is shown a schematic side view representation of a skincare mask 301 in accordance with an embodiment of the invention including a substrate 317. The skincare mask 301 is similar to that shown in FIGS. 1a and 1b, and comprises at least one humectant or hydrophilic layer 303 and at least one at least partially occlusive layer 305, but further comprises a substrate 317. The substrate 317 may be that as defined herein. The substrate is attached to one side of the at least one at least partially occlusive layer 305, while the at least one humectant or hydrophilic layer 303 is bonded to the other side of the the at least one at least partially occlusive layer 305. The at least one humectant or hydrophilic layer 303 is shown contacting a skin surface 319 of a user. In this way, the sequential dissolution of the at least one humectant or hydrophilic layer 303 followed by the at least one at least partially occlusive layer 305 can be effected (while being supported on the substrate 317).

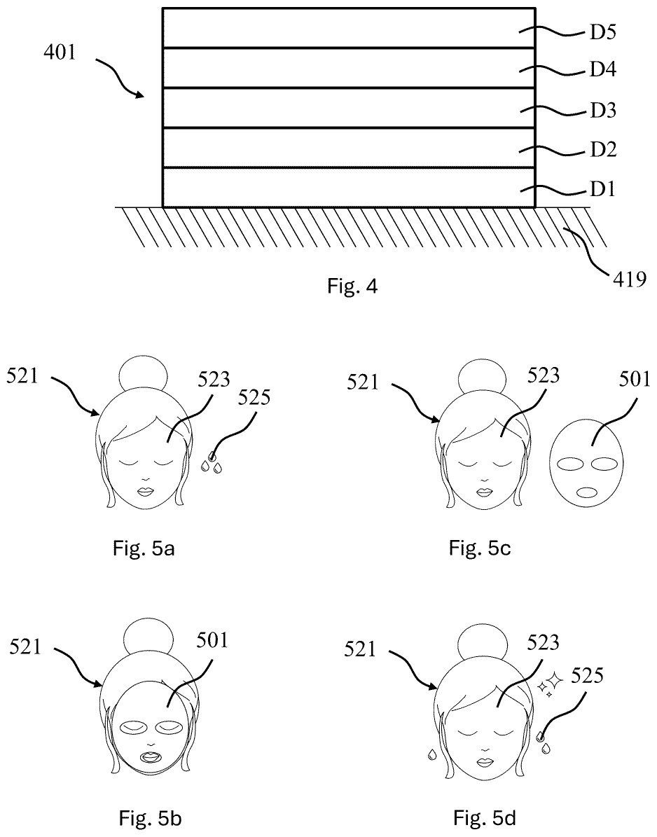

Referring now to FIG. 4, there is shown a schematic side view representation of a skincare mask 401 applied to a skin surface 419 of a user in accordance with an embodiment of the invention illustrating dissolution rates D1 to D5 of the different layers (wherein the skincare mask 401 is placed in contact with the skin surface 419 and allowed to dissolve without manual rubbing). D1 is the dissolution rate of the skincare mask 401 layer proximal (closest) the skin surface 419, while D5 is the dissolution rate of the skincare mask 401 layer distal (farthest) the skin surface 419. D1 and D2 may be 2-30 seconds each and D3 to D5 may be 5-60 seconds each.

Referring now to FIGS. 5A to 5d, there are shown schematic front view representations of a user 521 using a skincare mask 501 in accordance with an embodiment of the invention in which FIG. 5a shows a user's face 523 being moistened, FIG. 5b shows a user's face 523 applied with the skincare mask 501, FIG. 5c shows a user's face 523 with the skincare mask 501 removed, and FIG. 5d shows a user's face 523 optionally being rinsed. More particularly, there is illustrated a method of using a skincare mask 501 comprising at least one humectant or hydrophilic layer and at least one at least partially occlusive layer to cleanse skin (for instance, user's face 523), the method comprising the steps of: contacting the face 523 with moisture or water 525-FIG. 5a; placing the skincare mask 501 on the skin 523 with a humectant or hydrophilic layer facing the skin 523 and maintaining contact for a predetermined period to release skincare components while an at least partially occlusive layer reduces moisture loss-FIG. 5b; and removing the skincare mask 501 after partial or complete dissolution of one or more layers of the skincare mask 501-FIG. 5c. Optionally, after removal of the skincare mask 501, wiping or rinsing 525 the skin 523 to remove residue or impurities, or leaving residual components on the skin for extended contact, optionally including overnight use.

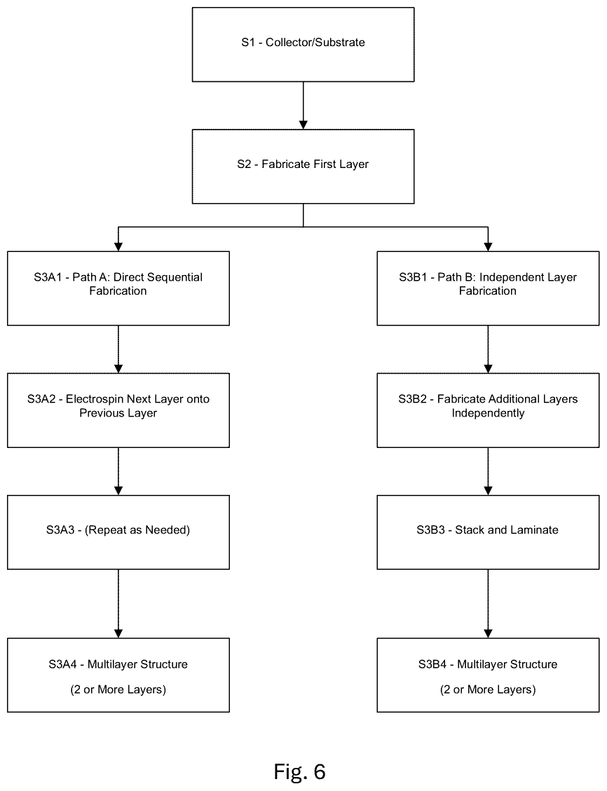

Referring now to FIG. 6, there is shown a flow diagram illustrating methods for fabricating a multilayer skincare mask in accordance with embodiments of the present invention. More particularly, there is disclosed a method of producing a skincare mask, comprising: fabricating at least one humectant or hydrophilic layer; fabricating at least one at least partially occlusive layer; and assembling the layers in a predefined spatial order such that the skincare mask provides the predetermined sequence of ingredients exposure to skin upon a single application. For instance, Step 1 (S1) is providing a Collector/Substrate; Step 2 (S2) is fabricating a First Layer; the next step can diverge with a first route being Step 3A1 (S3A1) Path A: Direct Sequential Fabrication, followed by Step 3A2 (S3A2) Electrospin Next Layer onto Previous Layer, followed by Step 3A3 (S3A3) repeat as needed, followed by Step 3A4 (S3A4) of realizing the multilayered skincare mask (2 or more layers); or a second route being Step 3B1 (S3B1) Path B: Independent Layer Fabrication, followed by Step 3B2 (S3B2) Fabricate Additional Layers Independently, followed by Step 3B3 (S3B3) Stack and Laminate, followed by Step 3B4 (S3B4) of realizing the multilayered skincare mask (2 or more layers). In either of the approaches (direct sequential layer-by-layer fabrication, such as by electrospinning-first route; or independent fabrication of individual layers followed by lamination-second route), the fabrication steps may be repeated to form any desired number of layers, resulting in a multilayer structure comprising two or more layers.

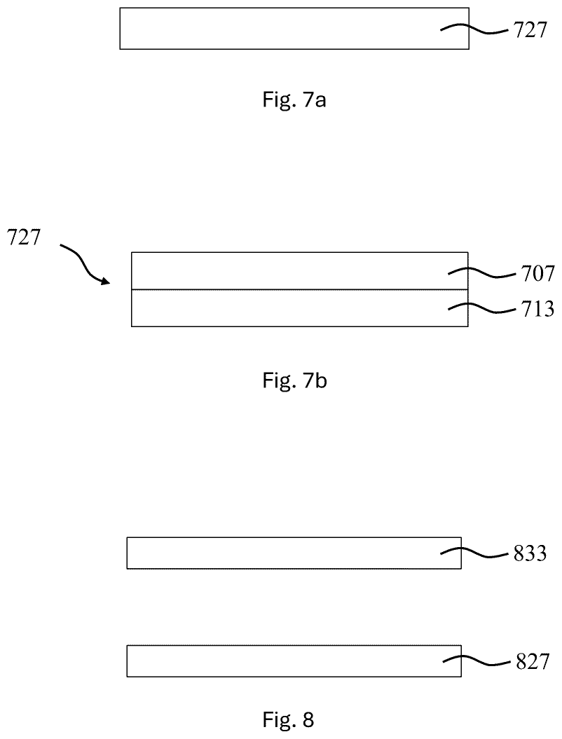

Referring now to FIG. 7a, there is shown a schematic side view representation of a skincare mask underlayer 727 in accordance with an embodiment of the invention, which, in this embodiment, comprises at least one humectant or hydrophilic layer.

Referring now to FIG. 7b, there is shown a schematic side view representation of a skincare mask underlayer 727 in accordance with another embodiment of the invention. In this embodiment, the skincare mask underlayer 727 comprises a toner layer 713 and a serum layer 707. The skincare mask underlayer 727 is inventive in its own right and allows a user to prime/hydrate their skin prior to applying a skincare mask (either one formed according to the present invention or a conventional skincare mask).

Referring now to FIG. 8, there is shown a schematic side view representation of a skincare mask underlayer 827 in accordance with an embodiment of the invention being configured/arranged relative to a skincare mask 833. The skincare mask underlayer 827 is beneath the skincare mask 833 so that, during use, the skincare mask underlayer 827 contacts the skin of a user's face first (before the skincare mask 833 makes contact with the skin of the user's face). In this way, the skin may be primed before the skincare mask 833 contacts the skin.

### EXAMPLES

The following examples were or will be carried out according to embodiments of the present disclosure.

### Example 1—Multilayer Skincare Mask

A skincare mask of the present disclosure can include a toning layer, an essence layer, a serum layer, and a moisturizing layer in that order, such that the toning layer contacts the skin first. Each layer of the skincare mask may be designed to dissolve into the skin prior to the next layer contacting the skin. The mask may be placed on the skin, such that the toning layer is closest to the skin and the moisturizing layer is the outermost layer. The toning layer may fully dissolve into the user's skin, allowing the essence layer to come in contact with the user's skin. The essence layer may then dissolve and allow the serum layer to contact the skin. The serum layer may then dissolve and allow the moisturizing layer to contact the skin. It is contemplated that each layer may dissolve in a time of about 5 to 30 seconds. In some embodiments, longer dissolution times for one or more layers are contemplated. Dissolution of the multilayer structure occurs rapidly upon contact with moisture, with multiple layers dissolving within approximately 10 seconds. The inventors understand that sequential dissolution (e.g, first layer fully or partially dissolves before a second layer contacts the skin and begins to dissolve) under these conditions can support staged dissolution based on differences in layer composition, morphology, and solubility.

### Example 2—Making a Multilayer Skincare Mask

Making the multilayer skincare mask of the present disclosure can include electrospinning each of the toning layer, the essence layer, the serum layer, and the moisturizing layer. Electrospinning can result in a skincare mask which has defined layers with no or minimal mixing at the interface of each layer. Making the skincare mask can further include fabricating one or more of the layers on a substrate, wherein the substrate serves as a base onto which the layers are electrospun.

### Prophetic Example: Differential Dissolution of Multilayer Sheet

A multilayer electrospun sheet comprising hydrophilic inner layers and more occlusive top layers may be formulated to exhibit staggered dissolution upon exposure to water. The sequential dissolution behavior is governed primarily by differences in material composition, rather than thickness alone, thereby enabling time-dependent release of active agents. Differential dissolution is achieved by selecting polymers with distinct hydrophilicity profiles. Materials such as hyaluronic acid, collagen, and formulations enriched with polyols (e.g., glycerin) can exhibit rapid dissolution due to their high aqueous solubility and strong affinity for water, whereas the occlusive or emollient-rich layers incorporate ingredients with relatively low water solubility (e.g., fatty alcohols, butters), resulting in slower dissolution.

A crosslinking agent or other dissolution-modifying additives may be incorporated into the occlusive-rich layer to delay its disintegration, allowing it to maintain structural integrity for an additional 5-60 seconds following hydration. In contrast, the hydrophilic layer is predicted to initiate dissolution within approximately 2-30 seconds. This sequential dissolution behavior allows staged exposure of different skincare actives, mimicking multi-step routines.

The hydrophilic inner layers may be formed with a thickness in a lower range (about 5 μm to about 150 μm) relative to one or more subsequent layers comprising semi-occlusive or occlusive ingredients (about 20 μm to about 300 μm), thereby contributing to faster dissolution of the hydrophilic layers and prolonged dissolution of the subsequent layers.

### Example: Electrospinning of Dissolving Nanofiber on a Silicone Backing

A flexible medical grade silicone substrate was used as the collector surface for electrospinning. A polymer solution was prepared containing hydrolyzed collagen (4.95 wt %), high-molecular-weight hyaluronic acid (0.37 wt %), ethanol (12.42 wt %), water (70.90 wt %), dimethylisosorbide (1.09 wt %), 1,3-propanediol (2.47 wt %), butylene glycol (1.66 wt %), dimethicone (0.26 wt %), heptyl glucoside (0.06 wt %), PPG-20 methyl glucose ether (0.59 wt %), and polyvinyl alcohol (PVA; 67,000 g/mol) at 5.21 wt %. The solution was loaded into a syringe fitted with a 21-gauge blunt-tip needle. Needle-based electrospinning parameters were as follows: voltage differential of 36 kV, flow rate of 1 mL/h, and a needle-to-substrate distance of 10 cm. The silicone substrate was mounted on a collector mandrel rotating at 8.6 rpm. The resulting supported sheet exhibited a smooth, conformable, and skin-compatible surface that facilitated uniform nanofiber deposition and convenient handling. The transparent silicone backing also enabled visualization of nanofiber dissolution.

### Example: Electrospinning of Dissolving Nanofiber on a Polypropylene Fabric Backing

A polypropylene nonwoven fabric was used as a temporary backing layer for electrospinning to provide mechanical support during fiber deposition and subsequent handling. A polymer solution was prepared containing hydrolyzed collagen (5.58 wt %), high-molecular-weight hyaluronic acid (0.28 wt %), low-molecular-weight hyaluronic acid (0.42 wt %), Centella asiatica extract (0.70 wt %), ethanol (13.95 wt %), water (65.97 wt %), polyvinyl alcohol (PVA; 66,000 g/mol) at 6.97 wt %, 1,3-propanediol (5.86 wt %), and allantoin (0.28 wt %). Needleless electrospinning was carried out using a rotating sprocket-wheel disc as the spinneret. The electrospinning parameters were as follows: a voltage of 45 kV, a spinneret tip-to-substrate distance of 12.5 cm, and a sprocket wheel rotation speed of 40 rpm. The polypropylene substrate was mounted on a collector plate. The resulting supported sheet provided a smooth, pliable surface that enabled straightforward handling.

### Prophetic Example: Fabrication of a Free Standing Nanofiber Mat Via Electrospinning

A polymer solution comprising water-soluble polymers and active ingredients was electrospun under controlled conditions onto a collector without the use of a supporting substrate. A nanofiber mat of sufficient thickness, ranging from approximately 30-200 μm, enabling the nanofiber layer to be self-supporting and mechanically stable upon removal from the collector. This self-supported configuration is suitable for applications requiring a dissolvable, conformable sheet mask capable of direct application to skin.

The present invention solves the problem of consumer burden in multi-step routines by spatially structuring actives into a single device mimicking the order and functional delivery of sequential skincare products. In its most basic form, the skincare delivery system comprises at least two primary types of layers: a humectant-rich layer and an emollient- and/or occlusive-rich layer. These layers can be further subdivided into multiple sublayers designed to incorporate additional active agents while maintaining their stability and preserving their potency through segregation within separate layers. When users apply the multilayered sheet to their wet skin, the nanofibers dissolve, allowing humectants to enhance moisture retention while the emollient and occlusive agents help slow moisture loss.

Traditionally, best practices for layering multiple skincare products prioritize enhancing skin hydration by maximizing water retention and barrier function, applying from the thinnest to the thickest product consistencies. There are three primary classes of moisturizing agents: humectants, occlusives, and emollients. Humectants function by attracting and binding water molecules, thereby reducing transepidermal water loss through evaporation. Emollients are oil-based compounds that soften the skin by forming a protective layer that helps retain moisture and minimize water loss. Occlusives are typically higher viscosity oil based ingredients that create a physical barrier on the skin surface to prevent transepidermal water loss.

Inadequate layering of multiple skincare products can lead to skin irritation and disruption of the skin barrier, particularly in individuals with sensitive or compromised skin. For example, applying retinol directly onto bare skin without proper buffering may result in localized redness or peeling due to excessive penetration of the active compound. Likewise, the premature application of emollient and/or occlusive-rich formulations prior to water-based moisturizers may diminish overall hydration efficacy, as the occlusive layer hinders the absorption of humectants and water-soluble actives. When humectants are used without an accompanying occlusive agent, they may promote increased transepidermal water loss, particularly from compromised skin in low-humidity environments. Such ineffective layering practices may therefore compromise both skin comfort and the overall efficacy of skincare routines.

It is well established that molecules exceeding approximately 500 Daltons in molecular weight exhibit limited ability to traverse the stratum corneum, the skin's primary barrier. The degree of lipophilicity of the active molecules affects their skin penetration performance. Penetration enhancing ingredients can improve the absorption of relatively hydrophilic active compounds into the deeper layers of the skin and enhance their potency. These enhancers operate via various mechanisms, including disruption of the stratum corneum lipid matrix, improvement of active compound solubility, and modification of skin protein structures to increase permeability.

To optimize the performance and user experience of multilayer skincare systems, it is essential to consider all relevant factors, such as molecular size, lipophilicity, active ingredient compatibility, and layering sequence, must be considered for the formulation and fabrication of the multilayer nanofibers. It is understood that no electrospun skincare products have incorporated a multilayer architecture designed to strategically separate and sequence active agents based on these considerations.

### Example Solution Formulation Recipes for Humectant-Rich Layers

The inner layers correspond to the skin-facing hydrophilic layer formulated to deliver rapid hydration and facilitate initial active deposition, priming the skin for subsequent actives in the subsequent layers. The intermediate layers contain complementary actives, humectants and emollients.

The active ingredients can be selected from small hydrophilic molecules capable of providing gentle exfoliation, promoting epidermal renewal, and imparting soothing or barrier-supporting effects. Such molecules may include, but are not limited to, lactic acid, mandelic acid, and gluconolactone, which aid in mild exfoliation and surface renewal, as well as soothing actives such as Centella asiatica extract and niacinamide, which help reduce irritation, support barrier repair, and improve overall skin tone. Additionally, bioactive peptides and nucleotides such as copper tripeptide, polydeoxyribonucleotide (PDRN), and hexapeptide-9 may be incorporated to promote skin regeneration, enhance collagen synthesis, and accelerate wound healing. Other examples of humectants include glycerin, sorbitol, trehalose, sodium PCA, or betaine. Examples of electrospun nanofiber recipes fabricated using different electrospinning techniques, including sprocket wheel and needle-based methods:

Hydrolyzed collagen 4.98 wt %, low-Mw hyaluronic acid 0.37 wt %, ethanol 12.44 wt %, water 71.30 wt %, polyvinyl alcohol (66000 g/mol) 5.23 wt %, dimethyl isosorbide 0.25 wt %, 1,3-propanediol 5.23 wt %, lactic acid 0.02 wt %, mandelic acid 0.17 wt %.

Hydrolyzed collagen 4.94 wt %, high-Mw hyaluronic acid 0.25 wt %, low-Mw hyaluronic acid 0.37 wt %, centella asiatica extract 0.12 wt %, ethanol 12.36 wt %, water 68.36 wt %, polyvinyl alcohol (66000 g/mol) 6.18 wt %, dimethyl isosorbide 0.87 wt %, 1,3-propanediol 5.19 wt %, niacinamide 1.24 wt %.

Hydrolyzed collagen 5.58 wt %, High-Mw hyaluronic acid 0.28 wt %, Low-Mw hyaluronic acid 0.42 wt %, centella asiatica extract 0.70 wt %, ethanol 13.95 wt %, water 65.97 wt %, polyvinyl alcohol (66000 g/mol) 6.97 wt %, 1,3-propanediol 5.86 wt %, allantoin 0.28 wt %.

Hydrolyzed collagen 5.64 wt %, high-Mw hyaluronic acid 0.28 wt %, low-Mw hyaluronic acid 0.42 wt %, ethanol 14.10 wt %, water 66.71 wt %, polyvinyl alcohol (66000 g/mol) 5.50 wt %, dimethyl isosorbide 0.99 wt %, 1,3-propanediol 5.92 wt %, bakuchiol 0.14 wt %, allantoin 0.28 wt %.

Hydrolyzed marine collagen 4.47 wt %, high-Mw hyaluronic acid 0.22 wt %, low-Mw hyaluronic acid 0.34 wt %, orange peel extract 0.22 wt %, ethanol 11.19 wt %, water 73.04 wt %, polyvinyl alcohol (66000 g/mol) 5.59 wt %, 1,3-propanediol 4.70 wt %, french pine extract 0.12 wt %, portulaca extract 0.11 wt %.

Hydrolyzed collagen 4.80 wt %, high-Mw hyaluronic acid 0.24 wt %, low-Mw hyaluronic acid 0.36 wt %, ethanol 12.01 wt %, water 68.82 wt %, polyvinyl alcohol (66000 g/mol) 6.01 wt %, dimethyl isosorbide 0.84 wt %, 1,3-propanediol 5.04 wt %, ascorbyl glucoside 1.63 wt %.

Hydrolyzed collagen 5.21 wt %, high-mw hyaluronic acid 0.39 wt %, ethanol 13.04 wt %, water 74.68 wt %, dimethylisosorbide 0.28 wt %, 1,3-propanediol 5.5 wt %, polyglycerin-10 0.9 wt %, polyvinyl alcohol (67000 g/mol) 5.48 wt %

Hydrolyzed collagen 4.86 wt %, high-Mw hyaluronic acid X0.36 wt %, low-Mw hyaluronic acid 0.36 wt %, ethanol 12.28 wt %, water 69.58 wt %, dimethylisosorbide 0.34 wt %, 1,3-propanediol 5.32 wt %, green tea extract 1.66 wt %, polyvinyl alcohol (67000 g/mol) 2.69 wt %, polyvinyl alcohol (130000 g/mol) 2.43 wt %.

Hydrolyzed collagen 4.89 wt %, high-Mw hyaluronic acid 0.36 wt %, ethanol X12.51 wt %, water 69.52 wt %, dimethylisosorbide 0.36 wt %, 1,3-propanediol 5.21 wt %, niacinamide 1.79 wt %, hydroxypropyl bis-hydroxyethyldimonium chloride 0.24 wt %, polyvinyl alcohol (67000 g/mol) 2.68 wt %, polyvinyl alcohol (130000 g/mol) 2.43 wt %.

Hydrolyzed collagen 5.71 wt %, high-Mw hyaluronic acid 0.57 wt %, low-Mw hyaluronic acid 0.86 wt %, ethanol 14.29 wt %, water 67.57 wt %, polyvinyl alcohol (67000 g/mol) 4.29 wt %, 1,3-propanediol 6.00 wt %, gotu kola extract 0.71 wt %.

Hydrolyzed collagen 6.67 wt %, high-Mw hyaluronic acid 0.67 wt %, low-Mw hyaluronic acid 1.00 wt %, ethanol 16.67 wt %, water 66.17 wt %, polyvinyl alcohol (67000 g/mol) 3.00 wt %, dimethyl isosorbide 5.00 wt %, gotu kola extract 0.83 wt %.

Hydrolyzed collagen 6.54 wt %, high-Mw hyaluronic acid 0.16 wt %, low-Mw hyaluronic acid 0.16 wt %, ethanol 16.86 wt %, water 67.59 wt %, polyvinyl alcohol (67000 g/mol) 1.50 wt %, dimethyl isosorbide 4.90 wt %, bifida ferment lysate 0.33 wt %, sorbitol 1.96 wt %.

### Example Formulation Recipes for Occlusive- and/or Emollient-Rich Layers

The outermost layer contains ingredients with occlusives, semi-occlusives and emollient to reduce evaporative loss and maintain a high-humidity microenvironment at the skin interface. An occlusive agent may be selected from hydrophobic or lipophilic molecules such as, olive oil, argan oil, ceramide, caprylyl methicone, squalane, coco-caprylate/caprate, and dimethicone. Examples of electrospun nanofiber recipes fabricated using different electrospinning techniques, including sprocket wheel and needle-based methods:

Hydrolyzed collagen 4.85 wt %, high-mw hyaluronic acid 0.24 wt %, low-mw hyaluronic acid 0.36 wt %, ethanol 12.13 wt %, water 69.52 wt %, polyvinyl alcohol (66000 g/mol) 6.07 wt %, 1,3-propanediol 5.10 wt %, lactic acid 1.70 wt %, γ-polyglutamic acid 0.02 wt %.

Hydrolyzed collagen 5.24 wt %, high-mw hyaluronic acid 0.38 wt %, ethanol 12.90 wt %, water 73.96 wt %, dimethylisosorbide 0.26 wt %, 1,3-propanediol 5.42 wt %, vegetable ceramide in glycerin 1.84 wt %, polyvinyl alcohol (67000 g/mol) 5.42 wt %

Hydrolyzed collagen 4.95 wt %, high-mw hyaluronic acid 0.37 wt %, ethanol 12.42 wt %, water 70.9 wt %, dimethylisosorbide 1.09 wt %, 1,3-propanediol 2.47 wt %, butylene glycol 1.66 wt %, dimethicone 0.26 wt %, heptyl glucoside 0.06 wt %, PPG-20 methyl glucose ether 0.59 wt %, polyvinyl alcohol (67000 g/mol) 5.21 wt %

Hydrolyzed collagen 4.89 wt %, high-mw hyaluronic acid 0.37 wt %, ethanol 12.26 wt %, water 70.14 wt %, dimethylisosorbide 1.1 wt %, 1,3-propanediol 2.45 wt %, butylene glycol 1.72 wt %, squalane 0.3 wt %, heptyl glucoside 0.67 wt %, PPG-20 methyl glucose ether 1.01 wt %, polyvinyl alcohol (67000 g/mol) 5.11 wt %

Hydrolyzed collagen 5.14 wt %, high-Mw hyaluronic acid 0.38 wt %, ethanol 12.89 wt %, water 73.22 wt %, polyvinyl alcohol (67000 g/mol) 5.37 wt %, dimethylisosorbide 1.1 wt %, 1,3-propanediol 5.45 wt %, argan oil 1.83 wt %

Hydrolyzed collagen 5.25 wt %, high-Mw hyaluronic acid 0.39 wt %, ethanol 12.96 wt %, water 74.02 wt %, polyvinyl alcohol (67000 g/mol) 5.42 wt %, dimethylisosorbide 0.72 wt %, 1,3-propanediol 5.52 wt %, jojoba oil 1.14 wt %

Hydrolyzed collagen 7.16 wt %, high-Mw hyaluronic acid 0.49 wt %, ethanol 12.54 wt %, water 68.41 wt %, polyvinyl alcohol (130000 g/mol) 2.39 wt %, dimethylisosorbide 1 wt %, 1,3-propanediol 5.36 wt %, vegetable ceramide in glycerin 1.76 wt %, heptyl glucoside 0.89 wt %

Hydrolyzed collagen 3.27 wt %, high-Mw hyaluronic acid 0.16 wt %, low-Mw hyaluronic acid 0.16 wt %, ethanol 17.42 wt %, water 69.28 wt %, polyvinyl alcohol (67000 g/mol) 2.52 wt %, dimethyl isosorbide 4.90 wt %, bakuchiol 0.33 wt %, PPG-20 methyl glucose ether 1.96 wt %.

Sunscreen protective properties may also be incorporated into the multilayered mask to provide photoprotection during use. Some examples include:

An electrospinning composition was prepared by combining 97.55 wt % PVA, 0.10 wt % avobenzone, 0.40 wt % octyl salicylate, 0.50 wt % octocrylene, 1.45 wt % hydroxypropyl beta cyclodextrin, and 0.10 wt % EDTA, in a solvent system comprising water, ethanol, butylene glycol, propylene diglycol, and hexanediol.

An electrospinning composition was prepared by combining 82.82% PVA, 0.4 wt % avobenzone, 1.6 wt % octyl salicylate, 2 wt % octocrylene, 5.82 wt % hydroxypropyl beta cyclodextrin and 0.10 wt % EDTA in a solvent system comprising water and ethanol.

### Prophetic Examples: Electrospun Formulation for Other Active-Loaded Delivery Layers

Hydrolyzed collagen 5.21 wt %, high-mw hyaluronic acid 0.39 wt %, ethanol 13.04 wt %, water 74.68 wt %, dimethyl isosorbide 0.28 wt %, 1,3-propanediol 5.5 wt %, copper tripeptide 0.02 wt %, polyvinyl alcohol (67000 g/mol) 5.48 wt %

Hydrolyzed collagen 4.80 wt %, high-Mw hyaluronic acid 0.24 wt %, low-Mw hyaluronic acid 0.36 wt %, ethanol 12.01 wt %, water 68.82 wt %, polyvinyl alcohol (66000 g/mol) 6.01 wt %, dimethyl isosorbide 0.84 wt %, hexapeptide-9 0.02 wt %, 1,3-propanediol 5.04 wt %

Hydrolyzed collagen 5.21 wt %, high-mw hyaluronic acid 0.39 wt %, ethanol 13.04 wt %, water 74.68 wt %, dimethyl isosorbide 0.28 wt %, 1,3-propanediol 5.5 wt %, polyvinyl alcohol (67000 g/mol) 5.48 wt %, polydeoxyribonucleotide 0.02 wt %

### Multilayer Fabrication Via Sequential Electrospinning

It is understood that although all layers rapidly hydrate, a transiently staggered release profile can be obtained under fast-dissolution conditions. The skin-facing layers, enriched in more hydrophilic and/or low molecular weight active ingredients preferentially contribute to the initial mass flux due to greater aqueous solubility and rapid dissolution at the skin interface. In contrast, more hydrophobic and/or higher molecular weight active ingredients localized in posterior layers exhibit slower matrix wetting and increased diffusional resistance.

### Example: Five Layer Formulations Fabricated Via Sequential Electrospinning to Provide Targeted Skincare Benefits for Nighttime Use. a Five-Layer Dissolvable Skincare Sheet to Emulate a Sequential Skincare Regimen: Toner→Serum 1→Serum 2→Eye Cream→Moisturizer Sequence

| | Layer 1 | | Layer 2 | | Layer 3 | | Layer 4 | | Layer 5 | |
|---|---|---|---|---|---|---|---|---|---|---|
| Polyvinyl alcohol (130,000 Da, DH = 88%) | 5.24 | wt % | 5.21 | wt % | 5.18 | wt % | 5.19 | wt % | 5.19 | wt % |
| Hydrolyzed Marine Collagen | 4.98 | wt % | 4.99 | wt % | 4.96 | wt % | 4.95 | wt % | 4.94 | wt % |
| High molecular weight hyaluronic acid | 0.4 | wt % | 0.38 | wt % | 0.38 | wt % | 0.37 | wt % | 0.37 | wt % |
| Centella Asiatica extract | — | | 0.34 | wt % | 0.34 | wt % | — | | — | |
| Azelaic acid | 0.04 | wt % | — | | — | | — | | — | |
| Green tea extract | — | | — | | 0.56 | wt % | — | | — | |
| Bakuchiol | — | | — | | — | | 0.13 | wt % | | |
| Vegetable ceramide | — | | — | | — | | — | | 0.6 | wt % |
| Ethanol | 12.48 | wt % | 12.4 | wt % | 12.33 | wt % | 13.33 | wt % | 12.65 | wt % |
| Water | 71.32 | wt % | 71.11 | wt % | 70.71 | wt % | 70.88 | wt % | 70.76 | wt % |
| Dimethylisosorbide | 0.3 | wt % | 0.27 | wt % | 0.26 | wt % | 0.23 | wt % | 0.27 | wt % |
| 1,3- Propanediol | 5.24 | wt % | 5.3 | wt % | 5.27 | wt % | 4.92 | wt % | 5.22 | wt % |

### Example: Four Layer Formulations Fabricated Via Sequential Electrospinning to Provide Targeted Skincare Benefits for Nighttime Use

| | Layer 1 | | Layer 2 | | Layer 3 | | Layer 4 | |
|---|---|---|---|---|---|---|---|---|
| Polyvinyl alcohol (67,000 Da, DH = 88%) | 2.68 | wt % | 2.69 | wt % | 5.21 | wt % | 5.11 | wt % |
| Polyvinyl alcohol (130,000 Da, DH = 88%) | 2.43 | wt % | 2.43 | wt % | — | | — | |
| Hydrolyzed Marine Collagen | 4.89 | wt % | 4.86 | wt % | 4.95 | wt % | 4.89 | wt % |
| High molecular weight hyaluronic acid | 0.36 | wt % | 0.36 | wt % | 0.37 | wt % | 0.37 | wt % |
| Niacinamide | 1.79 | wt % | — | | — | | — | |
| Hydroxypropyl bis-hydroxyethyldimonium chloride | 0.24 | wt % | — | | — | | — | |
| Green tea extract | — | | 1.66 | wt % | — | | — | |
| Bakuchiol | — | | — | | — | | — | |
| Dimethicone | — | | — | | 0.26 | wt % | — | |
| Squalane | — | | — | | — | | 0.3 | wt % |
| PPGl-20 methyl glucose ether | — | | — | | 0.59 | wt % | 1.01 | wt % |
| Heptyl glucoside | — | | — | | 0.06 | wt % | 0.67 | wt % |
| Ethanol | 12.51 | wt % | 12.28 | wt % | 12.42 | wt % | 12.26 | wt % |
| Water | 69.52 | wt % | 69.68 | wt % | 70.90 | wt % | 70.14 | wt % |
| Dimethylisosorbide | 0.36 | wt % | 0.34 | wt % | 1.09 | wt % | 1.1 | wt % |
| 1,3-Propanediol | 5.21 | wt % | 5.32 | wt % | 2.47 | wt % | 2.45 | wt % |

| | | | | | | |
|---|---|---|---|---|---|---|
| 1,3-Butylene glycol | — | — | 1.66 | wt % | 1.72 | wt % |

Prophetic example: a multilayered electrospun sheet mask is fabricated to provide targeted skincare benefits for daytime. A five-layer dissolvable skincare sheet to emulate a sequential skincare regimen: toner→serum 1→serum 2→essence→moisturizer sequence.

| | Layer 1 | | Layer 2 | | Layer 3 | | Layer 4 | | Layer 5 | |
|---|---|---|---|---|---|---|---|---|---|---|
| Polyvinyl alcohol (66,000 Da, | 5.23 | wt % | 6.01 | wt % | 6.18 | wt % | 6.97 | wt % | 6.07 | wt % |
| DH = 88%) | | | | | | | | | | |
| Hydrolyzed Marine Collagen | 4.98 | wt % | 4.80 | wt % | 4.94 | wt % | 5.58 | wt % | 4.85 | wt % |
| High molecular weight hyaluronic | — | | 0.24 | wt % | 0.25 | wt % | 0.28 | wt % | 0.24 | wt % |
| acid | | | | | | | | | | |
| Low molecular weight hyaluronic | 0.37 | wt % | 0.36 | wt % | 0.37 | wt % | 0.42 | wt % | 0.36 | wt % |
| acid | | | | | | | | | | |
| Lactic acid | 0.02 | wt % | — | | — | | — | | 1.70 | wt % |
| Mandelic acid | 0.17 | wt % | — | | — | | — | | — | |
| Ascorbyl glucoside | — | | 1.63 | wt % | — | | — | | — | |
| Centella asiatica extract | — | | — | | 0.12 | wt % | 0.70 | wt % | — | |
| Niacinamide | — | | — | | 1.24 | wt % | — | | — | |
| Allantoin | — | | — | | — | | 0.28 | wt % | — | |
| γ-Polyglutamic acid | — | | — | | — | | — | | 0.02 | wt % |
| Ethanol | 12.44 | wt % | 12.01 | wt % | 12.36 | wt % | 13.95 | wt % | 12.13 | wt % |
| Water | 71.30 | wt % | 68.82 | wt % | 68.36 | wt % | 65.97 | wt % | 69.52 | wt % |
| Dimethylisosorbide | 0.25 | wt % | 0.84 | wt % | 0.87 | wt % | 0.23 | wt % | — | |
| 1,3-Propanediol | 5.23 | wt % | 5.04 | wt % | 5.19 | wt % | 5.86 | wt % | 5.10 | wt % |

Example: lamination of five electrospun nanofiber sheets with specified formulations, followed by hydraulic pressing to form a multi-layered composite structure. Lamination was performed at 30° C. for 10 minutes using a manual heat press applying 0.5 tons-force pressure. Each layer of the skincare mask includes a base composition comprising polyvinyl alcohol (5.2-7 wt %, 66000 g/mol), hydrolyzed collagen (4.5-5.6 wt %), low-Mw hyaluronic acid (0.34-0.42 wt %), ethanol (11-14 wt %), water (66-73), propanediol (5.1-6 wt %).

| | Composition |
|---|---|
| Layer 1 | Dimethyl isosorbide (0.25 wt %), lactic acid 0.02 wt %, mandelic acid 0.17 wt %. |
| Layer 2 | High Mw hyaluronic acid (0.28 wt %), dimethyl isosorbide (0.23 wt %), centella |
| | asiatica extract (0.7 wt %), allantoin (0.28 wt %). |
| Layer 3 | High-Mw hyaluronic acid (0.28 wt %), dimethyl isosorbide 0.99 wt %, bakuchiol |
| | (0.14 wt %), allantoin (0.28 wt %). |
| Layer 4 | High-Mw hyaluronic acid (0.22 wt %), orange peel extract (0.22 wt %), french |
| | pine extract 0.12 wt %, portulaca extract 0.11 wt %. |
| Layer 5 | High-mw hyaluronic acid (0.24 wt %), lactic acid (1.70 wt %), γ-polyglutamic acid |
| | (0.02 wt %). |

### Unexpected Improvement in Fabrication Process Efficiency and Sheet Properties Through the Use of Electrospinning Promoter Co-Solvents.

Polyethylene oxide (PEO) is commonly used as an electrospinning promoter. However, its use in skincare formulations is undesirable due to the potential presence of dioxane impurities and its classification as a non-clean ingredient.

Unexpectedly, the incorporation of nonaqueous co-solvents such as 1,3-propanediol, 1,3-butylene glycol, DMI within a water-ethanol polymer system provided markedly improved electrospinning process stability or enhanced nanofiber property. It is understood that the inclusion of such co-solvents reduces the surface tension and viscosity of the polymer solution and additionally acts as a plasticizer, thereby enhancing polymer chain mobility and overall solution uniformity. These effects facilitate continuous fiber formation during electrospinning, leading to the production of a mechanically stable and homogeneous nanofiber sheet. In addition to serving as electrospinning promoters, the nonaqueous co-solvents deliver multifunctional skincare benefits, as humectants, emollients, and skin penetration enhancers.

TABLE 1
Comparison of electrospinnability with and without 1,3-propanediol or 1,3-butylene glycol.
The absence of nonaqueous alcohol resulted in a discontinuous electrospinning process.

| | | | | | | | Without | |
|---|---|---|---|---|---|---|---|---|
| | With 1,3- | | With 1,3- | | | | electrospinning | |
| | propanediol | | butylene glycol | | With DMI | | promoters | |
| Hydrolyzed marine collagen | 4.85 | wt % | 4.85 | wt % | 4.85 | wt % | 4.85 | wt % |
| High-Mw Hyaluronic acid | 0.24 | wt % | 0.24 | wt % | 0.24 | wt % | 0.24 | wt % |
| Low-Mw hyaluronic acid | 0.36 | wt % | 0.36 | wt % | 0.36 | wt % | 0.36 | wt % |
| Polyvinyl alcohol (66,000 | 6.07 | wt % | 6.07 | wt % | 6.07 | wt % | 6.07 | wt % |
| g/mol, DH = 88%) | | | | | | | | |
| Panthenol | 1.70 | wt % | 1.70 | wt % | 1.70 | wt % | 1.70 | wt % |
| γ-Polyglutamic Acid | 0.02 | wt % | 0.02 | wt % | 0.02 | wt % | 0.02 | wt % |
| Ethanol | 12.13 | wt % | 12.52 | wt % | 12.13 | wt % | 12.89 | wt % |
| Water | 69.52 | wt % | 71.68 | wt % | 69.52 | wt % | 73.87 | wt % |

| | | | | | |
|---|---|---|---|---|---|
| 1,3-Propanediol | 5.10 | wt % | — | — | — |

| | | | | | |
|---|---|---|---|---|---|
| 1,3-Butylene glycol | — | 2.55 | wt % | — | — |

| | | | | | |
|---|---|---|---|---|---|
| Dimethylisosorbide | — | — | 5.10 | wt % | — |

| | | | | |
|---|---|---|---|---|
| Electrospinnability | Continuous | Continuous | Continuous | Discontinuous |
| Nanofiber sheet property | <8 s | <10 s | <30 s | <15 s |

TABLE 2
Comparison of sheet property with and without 1,3-propanediol. In this formulation,
the absence of 1,3-propanediol resulted in undesirable nanofiber sheet properties.

| | With 1,3-propanediol | | Without 1,3-propanediol | |
|---|---|---|---|---|
| Hydrolyzed marine collagen | 4.98 | wt % | 4.98 | wt % |
| Lactic acid | 0.02 | wt % | 0.02 | wt % |
| Mandelic acid | 0.17 | wt % | 0.17 | wt % |
| Low-Mw hyaluronic acid | 0.37 | wt % | 0.37 | wt % |
| Polyvinyl alcohol (66,000 g/mol, | 5.23 | wt % | 5.23 | wt % |
| DH = 88%) | | | | |
| Ethanol | 12.44 | wt % | 13.27 | wt % |
| Water | 71.30 | wt % | 75.96 | wt % |

| | | | |
|---|---|---|---|
| Dimethylisosorbide | 0.25 | wt % | — |
| 1,3-Propanediol | 5.23 | wt % | — |

| | | |
|---|---|---|
| Electrospinnability | Continuous | Continuous |
| Nanofiber sheet property | <8 s dissolution time | Poor peelability from the |

substrate, yielding only small,
fragmented sections upon
removal.

TABLE 3
Comparison of sheet property with and without 1,3-propanediol. In this formulation,
the absence of 1,3-propanediol resulted in undesirable nanofiber sheet properties.

| | With 1,3-propanediol | | Without 1,3-propanediol | |
|---|---|---|---|---|
| Hydrolyzed marine collagen | 5.58 | wt % | 5.58 | wt % |
| Centella asiatica extract | 0.70 | wt % | 0.70 | wt % |
| Allantoin | 0.28 | wt % | 0.28 | wt % |
| High-Mw hyaluronic acid | 0.28 | wt % | 0.28 | wt % |
| Low-Mw hyaluronic acid | 0.42 | wt % | 0.42 | wt % |
| Polyvinyl alcohol (66,000 g/mol, | 6.97 | wt % | 6.97 | wt % |
| DH = 88%) | | | | |
| Ethanol | 13.95 | wt % | 14.98 | wt % |
| Water | 65.97 | wt % | 70.79 | wt % |

| | | | |
|---|---|---|---|
| 1,3-Propanediol | 5.86 | wt % | — |

| | | |
|---|---|---|
| Electrospinnability | Continuous | Continuous |
| Nanofiber sheet property | <5 s | <15 s dissolution time |

Exhibited brittleness below 20 C.

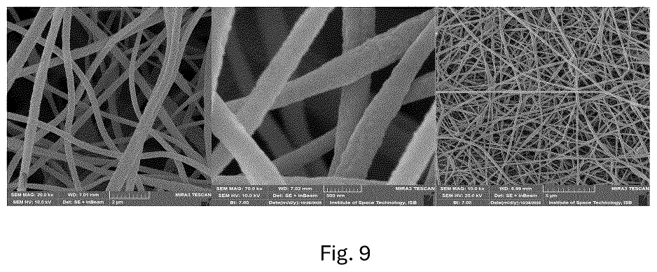

FIG. 9 is SEM images of electrospun nanofiber sheets containing 5-7 wt % PVA in the presence of 1,3-propanediol exhibited minimal to no bead formation and no observable fiber fusion. Another advantage of positioning the more hydrophobic layers (occlusive- and emollient-rich compositions) as the outer layers, fabricated first on a support layer or collector, is improved detachment of the electrospun mat from the substrate or collector. This configuration also mitigates removal difficulties that may occur when highly hydrophilic layers (humectant rich) are deposited directly onto the collector, wherein residual solvent can become trapped beneath a subsequently deposited hydrophobic layer.

TABLE 4
Effect of layer order on peelability from the substrate. In this example, the multilayered sheet
was fabricated onto an aluminum substrate. In a single-layer configuration, strong adhesion was
not observed, attributable to the ability of residual solvent to evaporate during storage. In
multilayer configurations, the layer order was critical for achieving good peelability. All electrospun
sheets were stored under pulled air by applying vacuum to a sealed bag overnight.

| | Deposited | | | | |
|---|---|---|---|---|---|
| | first (on | | | | Peelability |
| | Aluminum | Composition and | Deposited | Composition and | from |
| Configuration | substrate) | fabrication summary | last | fabrication summary | substrate |
| A | Occlusive- | Hydrolyzed collagen 4.95 | Humectant | Hydrolyzed collagen | Peeling was |
| | and | wt %, high-mw hyaluronic | layer | 5.21 wt %, high-mw | difficult, |
| | emollient- | acid 0.37 wt %, ethanol | | hyaluronic acid 0.39 | and the |
| | rich layer | 12.42 wt %, water 70.9 wt %, | | wt %, ethanol 13.04 wt %, | sheet |
| | | dimethylisosorbide 1.09 | | water 74.68 wt %, | fragmented |
| | | wt %, 1,3-propanediol 2.47 | | dimethylisosorbide | into flakes |
| | | wt %, butylene glycol 1.66 | | 0.28 wt %, 1,3- | as a result |
| | | wt %, dimethicone 0.26 | | propanediol 5.5 wt %, | of strong |
| | | wt %, heptyl glucoside 0.06 | | polyglycerin-10 0.9 wt %, | adhesion. |
| | | wt %, PPG-20 methyl | | polyvinyl alcohol | |
| | | glucose ether 0.59 wt %, | | (67000 g/mol) 5.48 wt % | |
| | | polyvinyl alcohol (67000 | | Voltage differential: 38 | |
| | | g/mol) 5.21 wt % | | kV | |
| | | Voltage differential: 36 kV | | Distance: 10 cm | |
| | | Distance: 10 cm | | Needle gauge: 22 Ga | |
| | | Needle gauge: 22 Ga | | Flow rate: 1 mL/hr | |
| | | Flow rate: 1 mL/hr | | | |
| B | Humectant | Hydrolyzed collagen 5.21 | Occlusive- | Hydrolyzed collagen | Peelable |
| (reverse | layer | wt %, high-mw hyaluronic | and | 4.95 wt %, high-mw | |
| order) | | acid 0.39 wt %, ethanol | emollient- | hyaluronic acid 0.37 | |
| | | 13.04 wt %, water 74.68 | rich | wt %, ethanol 12.42 wt %, | |
| | | wt %, dimethylisosorbide | layer | water 70.9 wt %, | |
| | | 0.28 wt %, 1,3-propanediol | | dimethylisosorbide 1.09 | |
| | | 5.5 wt %, polyglycerin-10 | | wt %, 1,3-propanediol | |
| | | 0.9 wt %, polyvinyl alcohol | | 2.47 wt %, butylene | |
| | | (67000 g/mol) 5.48 wt % | | glycol 1.66 wt %, | |
| | | Voltage differential: 36 kV | | dimethicone 0.26 wt %, | |
| | | Distance: 10 cm | | heptyl glucoside 0.06 | |
| | | Needle gauge: 22 Ga | | wt %, PPG-20 methyl | |
| | | Flow rate: 1 mL/hr | | glucose ether 0.59 wt %, | |
| | | | | polyvinyl alcohol | |
| | | | | (67000 g/mol) 5.21 wt % | |
| | | | | Voltage differential: 36 | |
| | | | | kV | |
| | | | | Distance: 10 cm | |
| | | | | Needle gauge: 22 Ga | |
| | | | | Flow rate: 1 mL/hr | |
| Control | Humectant | Hydrolyzed collagen 5.21 | — | — | Peelable |
| (single | layer | wt %, high-mw hyaluronic | | | |
| layer) | | acid 0.39 wt %, ethanol | | | |
| | | 13.04 wt %, water 74.68 | | | |
| | | wt %, dimethylisosorbide | | | |
| | | 0.28 wt %, 1,3-propanediol | | | |
| | | 5.5 wt %, polyglycerin-10 | | | |
| | | 0.9 wt %, polyvinyl alcohol | | | |
| | | (67000 g/mol) 5.48 wt % | | | |
| | | Voltage differential: 36 kV | | | |
| | | Distance: 10 cm | | | |
| | | Needle gauge: 22 Ga | | | |
| | | Flow rate: 1 mL/hr | | | |
| Control | Occlusive- | Hydrolyzed collagen 4.95 | — | — | Peelable |
| (single | and | wt %, high-mw hyaluronic | | | |
| layer) | emollient- | acid 0.37 wt %, ethanol | | | |
| | rich layer | 12.42 wt %, water 70.9 wt %, | | | |
| | | dimethylisosorbide 1.09 | | | |
| | | wt %, 1,3-propanediol 2.47 | | | |
| | | wt %, butylene glycol 1.66 | | | |
| | | wt %, dimethicone 0.26 | | | |
| | | wt %, heptyl glucoside 0.06 | | | |
| | | wt %, PPG-20 methyl | | | |
| | | glucose ether 0.59 wt %, | | | |
| | | polyvinyl alcohol (67000 | | | |
| | | g/mol) 5.21 wt % | | | |
| | | Voltage differential: 36 kV | | | |
| | | Distance: 10 cm | | | |
| | | Needle gauge: 22 Ga | | | |
| | | Flow rate: 1 mL/hr | | | |

TABLE 5
The comparative formulations demonstrate that electrospinning performance of
hyaluronic-acid-based solutions can be enhanced by incorporating moderately
polar, mid-volatility solvents, particularly cyclic esters such as γ-valerolactone,
in combination with moderately polar diols such as butylene glycol. Electrospinning
hyaluronic acid presents several challenges due to its high viscosity in aqueous
solutions, even at low concentrations. Hyaluronic acid often requires blending
with other polymers such as polyethylene oxide or polyvinyl alcohol, or the
use of less polar solvents like dimethylformamide (DMF). However, DMF is toxic,
which limits its suitability for skincare applications. While dimethyl isosorbide
and γ-valerolactone possess similar polarity and surface-tension ranges,
increasing the proportion of dimethyl isosorbide alone did not enable continuous
electrospinning. Similarly, increasing ethanol and heptyl glucoside to reduce
surface tension did not result in continuous fiber formation. In contrast,
the introduction of γ-valerolactone together with a moderately polar diol
produced an improvement, yielding continuous jet formation and a dry, non-
tacky fibrous web.

| | | | High-water + elevated | | | |
|---|---|---|---|---|---|---|
| | | | dimethyl isosorbide, | | γ-valerolactone/ | |
| | High water | | heptyl glucoside, and | | diol-containing | |
| | formulation | | ethanol formulation | | formulation | |
| High-Mw Hyaluronic | 0.64 | wt % | 0.56 | wt % | 0.62 | wr % |
| acid | | | | | | |
| Low-Mw hyaluronic | — | | — | | 0.13 | wt % |
| acid | | | | | | |
| Hydrolyzed marine | 7.59 | wt % | 6.72 | wt % | 7.36 | wt % |
| collagen | | | | | | |
| Heptyl glucoside | 0.35 | wt % | 0.83 | wt % | 0.34 | wt % |
| Dimethyl isosorbide | 0.53 | wt % | 1 | wt % | 2.43 | wt % |
| Ethanol | 12.57 | wt % | 21.56 | wt % | 9.87 | wt % |
| Water | 72.02 | wt % | 63.75 | wt % | 49.06 | wt % |
| 1,3-Propanediol | 5.8 | wt % | 5.13 | wt % | 2.49 | wt % |

| | | | | |
|---|---|---|---|---|
| 1,3-Butylene glycol | — | — | 2.92 | wt % |
| γ-valerolactone | — | — | 24.42 | wt % |

| Electrospinnability | Discontinuous | Discontinuous | Continuous |
|---|---|---|---|
| Nanofiber sheet | Wet, tacky deposit | Wet, tacky deposit | Dry, non-tacky |
| property | | | fibrous web |

TABLE 6
Specified concentration ranges for each ingredient class. Higher levels
of humectant are present in inner hydrophilic layers to allow for fast
dissolution upon contact with moist or wet skin. The outer layers can
accommodate higher levels of occlusives and emollients, which provide
barrier function while reducing tack and improving peelability.

| | Inner layers | | Outer layers (at least semi- | |
|---|---|---|---|---|
| | (humectant rich) | | occlusives and/or emollient rich) | |
| Carrier polymer | 0.5-20% | | 0.5-20% | |
| Co-polymer/polymer blend | 0-15 | wt % | 0-15 | wt % |
| component | | | | |
| Humectant | 0.001-20% | | 0-15 | wt % |
| Emollient | 0-5 | wt % | 0.001-20 | wt % |
| Occlusive/Semi-occlusive | 0-5 | wt % | 0.001-20 | wt % |
| Co-solvent electrospinning | 0.001-10 | wt % | 0.001-10 | wt % |
| enhancer/penetration enhancer | | | | |

In addition to the polymers and solvents specifically exemplified herein, the composition may include other suitable water-soluble or biodegradable polymers, including but not limited to polyvinylalcohol, polyvinylpyrrolidone, polyethylene oxide, poly(2-ethyl-2-oxazoline), pullulan, cellulose derivatives such as hydroxypropyl methylcellulose, chitosan, gelatin, polycaprolactone, and their copolymers, derivatives, or blends.

Suitable solvents may include, without limitation, ethanol, isopropanol, propanediol, butylene glycol, hexanediol, pentanediol isomers, isopentyldiol, methylpropanediol, dipropylene glycol, ethoxydiglycol, and caprylyl glycol, glycerin, ethylhexylglycerin, isopropylidenglycerin, dimethyl isosorbide and propylene carbonate. These solvents are selected for their compatibility with the polymers and active ingredients and their ability to facilitate electrospinning. Some of these low to moderate volatility solvents may additionally function as penetration enhancers, for example 1,3-propanediol, butylene glycol, dimethyl isosorbide, and ethoxydiglycol, while also serving roles such as plasticizers, humectants, or viscosity modifiers.

Franz diffusion cell studies, along with Corneometer and Tewameter assessments, are planned to evaluate and confirm the penetration enhancement properties and skin hydration and barrier function effects of these co-solvents and active ingredients. These ongoing and future studies are intended to support the utility and mechanism of action of the present formulations.

### Example: Electrospinning of Dissolving Nanofiber on a Silicone Backing

A flexible medical grade silicone substrate was used as the collector surface for electrospinning. A polymer solution was prepared containing hydrolyzed collagen (4.95 wt %), high-molecular-weight hyaluronic acid (0.37 wt %), ethanol (12.42 wt %), water (70.90 wt %), dimethylisosorbide (1.09 wt %), 1,3-propanediol (2.47 wt %), butylene glycol (1.66 wt %), dimethicone (0.26 wt %), heptyl glucoside (0.06 wt %), PPG-20 methyl glucose ether (0.59 wt %), and polyvinyl alcohol (PVA; 67,000 g/mol) at 5.21 wt %. The solution was loaded into a syringe fitted with a 21-gauge blunt-tip needle. Needle-based electrospinning parameters were as follows: voltage differential of 36 kV, flow rate of 1 mL/h, and a needle-to-substrate distance of 10 cm. The silicone substrate was mounted on a collector mandrel rotating at 8.6 rpm. The resulting supported sheet exhibited a smooth, conformable, and skin-compatible surface that facilitated uniform nanofiber deposition and convenient handling. The transparent silicone backing also enabled visualization of nanofiber dissolution.

### Example: Electrospinning of Dissolving Nanofiber on a Polypropylene Fabric Backing

A polypropylene nonwoven fabric was used as a temporary backing layer for electrospinning to provide mechanical support during fiber deposition and subsequent handling. A polymer solution was prepared containing hydrolyzed collagen (5.58 wt %), high-molecular-weight hyaluronic acid (0.28 wt %), low-molecular-weight hyaluronic acid (0.42 wt %), Centella asiatica extract (0.70 wt %), ethanol (13.95 wt %), water (65.97 wt %), polyvinyl alcohol (PVA; 66,000 g/mol) at 6.97 wt %, 1,3-propanediol (5.86 wt %), and allantoin (0.28 wt %). Needleless electrospinning was carried out using a rotating sprocket-wheel disc as the spinneret. The electrospinning parameters were as follows: a voltage of 45 kV, a spinneret tip-to-substrate distance of 12.5 cm, and a sprocket wheel rotation speed of 40 rpm. The polypropylene substrate was mounted on a collector plate. The resulting supported sheet provided a smooth, pliable surface that enabled straightforward handling.

### Prophetic Example: Fabrication of a Free Standing Nanofiber Mat Via Electrospinning

A polymer solution comprising water-soluble polymers and active ingredients was electrospun under controlled conditions onto a collector without the use of a supporting substrate. A nanofiber mat of sufficient thickness, ranging from approximately 30-200 μm, enabling the nanofiber layer to be self-supporting and mechanically stable upon removal from the collector. This self-supported configuration is suitable for applications requiring a dissolvable, conformable sheet mask capable of direct application to skin.

### Prophetic Example: Differential Dissolution of Multilayer Sheet

A multilayer electrospun sheet comprising hydrophilic inner layers and more occlusive top layers may be formulated to exhibit staggered dissolution upon exposure to water. The sequential dissolution behavior is governed primarily by differences in material composition, rather than thickness alone, thereby enabling time-dependent release of active agents. In some embodiments, differential dissolution is achieved by selecting polymers with distinct hydrophilicity profiles. Materials such as hyaluronic acid, collagen, and formulations enriched with polyols (e.g., glycerin) can exhibit rapid dissolution due to their high aqueous solubility and strong affinity for water, whereas the occlusive or emollient-rich layers incorporate ingredients with relatively low water solubility (e.g., fatty alcohols, butters), resulting in slower dissolution. In certain embodiments, a crosslinking agent or other dissolution-modifying additives may be incorporated into the occlusive-rich layer to delay its disintegration, allowing it to maintain structural integrity for an additional 5-60 seconds following hydration. In contrast, the hydrophilic layer is predicted to initiate dissolution within approximately 2-30 seconds. This sequential dissolution behavior allows staged exposure of different skincare actives, mimicking multi-step routines.

### Prophetic Example: Expected Hydration Benefit of Humectant-Occlusive Multilayer Electrospun Sheet

Corneometer measurements are expected to demonstrate enhanced skin hydration when the humectant-rich and occlusive-rich layers are delivered sequentially via the multilayer electrospun mask, as compared to application of a single-layer control.

This disclosure is not limited to the particular systems, devices and methods described, as these may vary. The terminology used in the description is for the purpose of describing the particular versions or embodiments only and is not intended to limit the scope.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 compounds refers to groups having 1, 2, or 3 compounds. Similarly, a group having 1-5 cells refers to groups having 1, 2, 3, 4, or 5 compounds, and so forth.

Various of the above-disclosed and other features and functions, or alternatives thereof, may be combined into many other different systems or applications. Various presently unforeseen or unanticipated alternatives, modifications, variations, or improvements therein may be subsequently made by those skilled in the art, each of which is also intended to be encompassed by the disclosed embodiments.

What is claimed is:

1. A skincare mask comprising at least one humectant or hydrophilic layer and at least one at least partially occlusive layer, wherein the at least one humectant or hydrophilic layer and the at least one at least partially occlusive layer are arranged in a predefined spatial order that structurally encodes a predetermined sequence of ingredient exposure to skin upon a single application of the skincare mask.

2. The skincare mask of claim 1, wherein the predetermined sequence of ingredient exposure is independent of user application behavior due to the predefined spatial order of the layers and their differential dissolution behavior upon application.

3. The skincare mask of claim 1, wherein the at least one humectant or hydrophilic layer is positioned closer to a skin-contacting surface of the skincare mask than the at least one at least partially occlusive layer.

4. The skincare mask of claim 1, wherein the at least one at least partially occlusive layer is positioned outwardly relative to the at least one humectant or hydrophilic layer.

5. The skincare mask of claim 1, wherein the at least one humectant or hydrophilic layer and the at least one partially occlusive layer have different compositions.

6. The skincare mask of claim 1, wherein the at least one humectant or hydrophilic layer and the at least one at least partially occlusive layer have different dissolution behaviors upon exposure to moisture.

7. The skincare mask of claim 1, wherein the layers are configured to physically separate ingredients that are chemically or physically incompatible when co-formulated, by isolating such ingredients in separate layers prior to use.

8. The skincare mask of claim 1, wherein the layers comprise physically discrete layers.

9. The skincare mask of claim 8, wherein the at least one humectant or hydrophilic layer and at least one at least partially occlusive layer comprise nanofiber layers.

10. The skincare mask of claim 9, wherein the nanofiber layers are electrospun nanofiber layers.

11. The skincare mask of claim 1, wherein the at least one humectant or hydrophilic layer is configured to dissolve more rapidly upon application to skin than the least one at least partially occlusive layer.

12. The skincare mask of claim 1, wherein the at least one humectant or hydrophilic layer is configured to attract and bind water molecules for increasing skin hydration upon application.

13. The skincare mask of claim 1, wherein the at least one at least partially occlusive layer is configured to reduce transepidermal water loss (TEWL) by forming a temporary barrier on the skin surface upon application.

14. The skincare mask of claim 1, wherein the skincare mask comprises two primary functional layers, including: a humectant-rich layer for use in skin hydration; and an emollient and/or at least partially occlusive-rich layer for use in skin barrier protection.

15. The skincare mask of claim 1, wherein the skincare mask comprises at least four functional layers, including: a toning layer; an essence layer; a serum layer; and a moisturizer layer.

16. The skincare mask of claim 1, wherein at least one of the layers further comprises an additive configured to improve electrospinning continuity, nanofiber mechanical integrity, and dissolution behavior.

17. The skincare mask of claim 16, wherein the additive comprises at least one penetration enhancer configured to modify solution properties during fabrication and/or nanofiber matrix properties after fabrication for improving electrospinning continuity, nanofiber uniformity, mechanical integrity, and dissolution behavior of layers.

18. The skincare mask of claim 1, wherein the at least one humectant or hydrophilic layer comprises at least one of a toning layer, an essence layer, or a serum layer.

19. The skincare mask of claim 1, wherein the at least one partially occlusive layer comprises at least one of a serum layer or a moisturizing layer.

20. The skincare mask of claim 1, wherein the at least one humectant or hydrophilic layer is substantially a hydrating layer.

21. The skincare mask of claim 1, wherein the at least one partially occlusive layer comprises at least some hydrophobic material.

22. The skincare mask of claim 1, wherein the at least one humectant or hydrophilic layer comprises about 0.5-20 wt % of carrier polymer, about 0-15 wt % of co-polymer and/or polymer blend component, about 0.001-20 wt % of humectant, about 0-5 wt % of at least partially occlusive material, about 0.001-10 wt % of co-solvent electrospinning enhancer and/or penetration enhancer; and about 0-5 wt % of emollient.

23. The skincare mask of claim 1, wherein the at least one partially occlusive layer comprises about 0.5-20 wt % of carrier polymer, about 0-15 wt % of co-polymer and/or polymer blend component, about 0-15 wt % of humectant, about 0.001-20 wt % of at least partially occlusive material, about 0.001-10 wt % of co-solvent electrospinning enhancer and/or penetration enhancer; and about 0.001-20 wt % of emollient.

24. A method of producing a skincare mask, comprising: fabricating at least one humectant or hydrophilic layer; fabricating at least one at least partially occlusive layer; and assembling the layers in a predefined spatial order such that the skincare mask provides the predetermined sequence of ingredients exposure to skin upon a single application.

25. The method of claim 24, wherein fabricating comprises electrospinning, electrospraying, solvent casting, coating, freeze drying, hot melt extrusion, 3D printing, or combinations thereof.

26. The method of claim 24, comprising fabricating at least four functional layers, including a toning layer, an essence layer, a serum layer, and a moisturizing layer.

27. The method of claim 24, further comprising bonding the layers for preserving the predefined spatial order.

28. A method of producing a skincare mask, comprising: fabricating a first layer comprising a toner; fabricating a second layer comprising an essence; fabricating a third layer comprising a serum; and fabricating a fourth layer comprising a moisturizer.
